(19) United States
(12) Patent Application Publication
(10) Pub. No.: US 2021/0271291 A1
(43) Pub. Date: Sep. 2, 2021

(54) DOCKING STATION SYSTEM

(71) Applicant: DTHERA SCIENCES OPERATIONS, INC., San Diego, CA (US)

(72) Inventors: David KEENE, San Diego, CA (US); Brian John KANG, San Diego, CA (US); Richard Arthur SPINDEL, San Diego, CA (US); Benjamin DOLGIN-GARDNER, San Diego, CA (US); Thomas WALKER, San Diego, CA (US); Rebecca Emily SENGER, San Diego, CA (US); Yevgeniy KOSTIKOV, San Diego, CA (US); Edward Michael COX, San Diego, CA (US)

(21) Appl. No.: 17/253,089

(22) PCT Filed: Jun. 21, 2019
(86) PCT No.: PCT/US19/38583

§ 371 Date: Dec. 16, 2020

Related U.S. Application Data
(60) Provisional application No. 62/689,004, filed on Jun. 22, 2018.

(51) Int. Cl. G06F 1/16; H01R 13/24; H01R 13/62; G16H 20/70; H04B 1/3877

(52) U.S. Cl. CPC G06F 1/1632; H01R 13/24; H04B 1/3877; G16H 20/70; H01R 13/6205

(57) ABSTRACT

An electronic docking system includes a dock comprising a recess having at least one vertically oriented side wall and a recess bottom face, wherein the dock comprises a pogo pin connector extending from the recess bottom face; a first protrusion extending in a substantially perpendicular direction from the recess bottom face and a second protrusion extending in a substantially perpendicular direction from the recess bottom face; and an electronic device includes an electrical contact configured to contact the pogo pin connector; a first alignment slot configured to accept the first protrusion and a second alignment slot configured to accept the second protrusion, wherein at least a portion of the exterior perimeter of the recess comprises a rounded or beveled edge.

### CROSS-REFERENCE

This application claims the benefit of U.S. Provisional Application No. 62/689,004, filed Jun. 22, 2018, which is hereby incorporated by reference in its entirety herein.

### BACKGROUND OF THE INVENTION

Estimates indicate that there are more than 5 million Americans suffering from Alzheimer's disease (AD) and other dementias. The cost of care for dementia patients is expected to quadruple from $277 billion in 2018 to more than $1.1 trillion in 2050. Dementia is also a growing problem for the military, and veterans with traumatic brain injury (TBI) in particular have been shown to have an increased risk of developing dementia.

According to the Alzheimer's Association, Alzheimer's dementia “is the only top 10 cause of death that cannot be prevented, cured or even slowed.” The cognitive deficits and behavioral symptoms (e.g., depression, anxiety, and apathy) are difficult to treat in AD and other forms of dementia. Currently the first-line of treatment for AD are pharmaceuticals, but these have met with only limited success, with many high-profile AD drugs having disappointing results. For this reason, there has been a renewed focus on non-pharmaceutical approaches that can target mood and physiological distress, rather than on pharmaceutical approaches.

Reminiscence therapy (RT) is behavioral intervention that involves the introduction of familiar pictures, music, or other materials to help individuals reminisce about their past experiences. RT is the most commonly used non-pharmacological therapy in AD and other types of dementia, and has been used since the 1960s for home care and in the nursing home and hospital settings.

RT has been shown in clinical studies to have a positive impact on mood and cognitive performance in individuals with Alzheimer's or other dementias, as well as in older adults with social isolation. Specifically, RT has been found to reduce depression, apathy, and loneliness, while improving cognitive performance, behavioral functioning, mood, communication, interaction, quality of life, life satisfaction, well-being, self-esteem, activities of daily living, and social activities. A recent meta-analysis of 12 randomized controlled studies demonstrated that RT significantly improved cognitive function and reduced depression in dementia, and concluded that RT should be considered as routine care for those with dementia, particularly for patients in memory care facilities. A recent systematic review of the literature concluded that RT has the potential to improve quality of life, cogitative function, communication, and mood, but also stated that the literature is hard to compare because the methods of delivery of RT have been highly variable.

A major limitation of RT is that it must be provided physically by a human caregiver. For this reason, it is highly labor-intensive, repetitive, and time-consuming. It is simply not practical for a family caregiver to sit with a dementia patient to go over the same photo scrapbook, home videos, or music on a daily basis, and using a therapist or professional therapist for frequent RT is cost prohibitive. Therefore, RT is usually given sporadically and in formal group therapy sessions. Furthermore, RT is given when it is convenient to the caregiver, not at the convenience of the patient, further limiting the consistency of its use. Although digital RT technologies are now under development that may facilitate delivery of RT to individual patients, none are scalable and none have yet been widely adopted. Hence there is a need for improved digital RT technologies that are scalable and that can be provided frequently, consistently, and without the need to depend on family members or healthcare providers.

### SUMMARY OF THE INVENTION

Described herein is an online-based story-sharing platform. This unique approach to RT allows users to record audio over photos as a way to share memories with family members who are suffering from a neurological or psychiatric condition. This platform is a potential digital therapeutic that allows patients to reminisce about their past, but does not have the structured time requirement or one-on-one administration that is needed with these formal therapies. Furthermore, as an online-based story-sharing platform, it is readily accessible and can easily be used on an ongoing basis by patients.

The technology is unique in that it allows multiple family members, even if they are separated by time and place, to collaborate on the stories in just a few minutes a day. The platform transforms the short audio notes and individual photos into rich documentary-like stories that are then archived in a private and secured database. These stories can then be viewed easily with a tablet whenever the patient chooses and the interface is simple to operate. The tablet and docking station technology has the potential to be a practical and highly implementable adjunct behavioral intervention for a variety of patients, including those with dementia.

In addition to the many benefits provided to the patient, the tablet and docking station technology is also expected to benefit the caregiver. This technology enables family members to engage with caregivers from anywhere in the world on their own time. This reduces caregiver burden by making it easier for additional family members, friends or healthcare providers to become involved in the care of a patient remotely. It also reduces the guilt associated with not being directly involved in care, and at the same time provides a caregiver with satisfaction of being notified of positive emotional feedback from stories shared directly by that caregiver.

Unfortunately, pharmaceutical approaches to Alzheimer's Disease have not been particularly successful, with many high profile drugs having disappointing results. For this reason, there has been a renewed focus on non-pharmaceutical approaches, such as exercise, diet, and cognitive training programs, that can target mood and physiological distress, rather than on pharmaceutical approaches that slow the progression of cognitive deficits. Various non-pharmacological approaches have been developed or are under development, with limited acceptance to date. Related digital senior care products include personalized music platforms, virtual reality platforms to transport seniors to relive prior memories, and kiosk and desktop-based solutions for use in senior care facilities. Other related products focus on family engagement, memory assessment, caregiver support and coaching, and technology simplification.

The tablet and docking station technology solution described herein is just one of the unique platforms among digital reminiscence therapy platforms, in that, when combined with digital software and cloud-based counterparts, they personalize RT for the patient into custom stories, and can optimize content being delivered to the patient using machine learning, proprietary emotional recognition software, as well as feedback from the family. This level of technology allows for an automatic process for personalized therapy, optimizing potential response from patients. Further, it utilizes and encourages family engagement, providing benefit to the caregiver as well as the patient. In addition, the solution is a truly scalable solution, in that it is a low-cost platform only relying on a custom tablet, family members' existing mobile devices, and the cloud. Therefore, this technology can be used at any home or senior care/memory care facility, and in rural and isolated settings where the purchase of large shared kiosks or VR equipment, or specialized staff or caregiver training, may not be practical.

It is well known that elderly people have greater difficulty than others in using computers and technology in general, and that demographic variables and cognitive abilities influence older adults' ability to use computers and other technologies. This problem is exacerbated when the end user has dementia. Therefore a key feature of the tablet and docking station technology described herein is that it is simple to setup, remove from the docking station, use, turn off, replace on the docking station, and charge.

Due to its simple user interface, a patient's openness to new technology is also not a concern (as it is with many other solutions). Patients are given a tablet with no menus or buttons; a patient simply picks up the tablet to begin viewing stories. Overcoming the possible fear of new technology by a patient is almost entirely circumvented.

The system features three key components: (1) an app that can be downloaded onto one or more family members' phones; (2) a cloud-based server; and (3) a tablet with a docking station that is used by the patient. The purpose of the system is to move stories from the family to the patient. The stories are moved through the cloud-based server, which runs on a secure cloud service platform, offering compute power, database storage, content delivery and other functionality primarily for businesses. The responsibility of the family app and the server together is to request story info via a custom AI chatbot—the AI chatbot chats with family members and directs them to upload photos and tell audio stories. Requested photos are based on themes such as weddings, vacations and birthdays, and when recording audio, the user has the option to speak freely or use prewritten scripts to help shape the story.

Provided herein is an electronic docking system comprising: a dock comprising a recess having at least one vertically oriented side wall and a recess bottom face, wherein the dock comprises: a pogo pin connector extending from the recess bottom face; a first protrusion extending in a substantially perpendicular direction from the recess bottom face; and a second protrusion extending in a substantially perpendicular direction from the recess bottom face; and an electronic device: an electrical contact configured to contact the pogo pin connector; a first alignment slot configured to accept the first protrusion; and a second alignment slot configured to accept the second protrusion, wherein at least a portion of the exterior perimeter of the recess comprises a rounded or beveled edge. In some embodiments, the electronic docking system further comprises at least one of one or more magnets within the recess and one or more magnets inside or coupled with the electronic device and configured to mate with the one or more magnets in the recess. In some embodiments, the electronic docking system, the electronic device comprises a first alignment slot configured to accept a protrusion extending from the first protrusion. In some embodiments, the electronic docking system, the electronic device comprises a first and second alignment slot configured to accept a first and a second protrusion extending from the first and second protrusion. In some embodiments, the dock further comprises at least one of an indicator, a motion sensor, and an ambient light sensor. In some embodiments of the electronic docking system, the electronic device further comprises at least one of an indicator, a motion sensor, and an ambient light sensor. In some embodiments, the electronic device comprises laptop computers, notebook computers, sub-notebook computers, netbook computers, netpad computers, media streaming devices, handheld computers, Internet appliances, mobile smartphones, tablet computers, disposable tablet computers, personal digital assistants, and video game consoles. Suitable tablet computers include those with booklet, slate, and convertible configurations, known to those of skill in the art. In some embodiments of the electronic docking system, the pogo pin connector comprises 5 pogo pins. In some embodiments of the electronic docking system, the dock further comprises at least one support protrusion extending vertically from a perimeter edge of the recess of the dock. In some embodiments, the indicator indicates a charging mode corresponding to a transfer of energy from the pogo pin connector to the electronic device. In some embodiments, the indicator is further activated or deactivated by at least one of: the motion sensor and the ambient light sensor. In some embodiments, the recess in the dock is generally rectangular and wherein the at least one vertically oriented side wall comprises four side walls. In some embodiments, the at least one vertically oriented side wall in the recess of the dock is tapered away from at least one of the first protrusion and the second protrusion. In some embodiments, the recess of the dock is configured to guide an insertion of an edge of the electronic device such that the electrical contact of the electronic device accurately aligns with the pogo pin connector of the dock. In some embodiments, the recess of the dock is configured to guide an insertion of an edge of the electronic device such that the alignment slot of the electronic device accurately aligns with a protrusion extending from the recess bottom face. In some embodiments, at least one of the first protrusion and the second protrusion is oblong in shape.

Provided herein is a docking station for an electronic system comprising: a recess having at least one vertically oriented side wall and a recess bottom face; a pogo pin connector extending from the recess bottom face; a first protrusion extending in a substantially perpendicular direction from the recess bottom face; a second protrusion extending in a substantially perpendicular direction from the recess bottom face; and at least one support protrusion configured to provide support to an electronic device when placed in the recess of the docking station.

In some embodiments, the docking station further comprises at least one magnet within the recess configured to mate with one or more corresponding magnets inside or coupled with the electronic device when the electronic device is placed in the recess. In some embodiments, the docking station further comprises at least one of an indicator, a motion sensor, and an ambient light sensor. In some embodiments, the motion sensor is a combination sensor also configurable as an ambient light sensor. In some embodiments, the indicator is further activated or deactivated by at least one of: the motion sensor and the ambient light sensor. In some embodiments, the pogo pin connector comprises 5 pogo pins. In some embodiments, the at least one support protrusion is configured to provide support to an electronic device when placed in the recess of the docking station. In some embodiments, the indicator indicates a charging mode corresponding to a transfer of energy from the pogo pin connector to the electronic device. In some embodiments, the recess is generally rectangular and wherein the at least one vertically oriented side wall comprises four side walls. In some embodiments, the at least one vertically oriented side wall in the recess of the dock is tapered away from at least one of the first protrusion and the second protrusion. In some embodiments, the recess of the dock is configured to guide an insertion of an edge of the electronic device such that the electrical contact of the electronic device accurately aligns with the pogo pin connector of the dock. In some embodiments, the recess of the dock is configured to guide an insertion of an edge of the electronic device such that the alignment slot of the electronic device accurately aligns with a protrusion extending from the recess bottom face. In some embodiments, at least one of the first protrusion and the second protrusion is oblong in shape. Provided herein is an electronic docking system comprising: a dock comprising a recess having at least one vertically oriented side wall and a recess bottom face, wherein the dock comprises: a pogo pin connector extending from the recess bottom face; a first protrusion extending in a substantially perpendicular direction from the recess bottom face; and an electronic device having a device width and a device length: an electrical contact configured to contact the pogo pin connector; a first alignment slot configured to accept the first protrusion, wherein at least a portion of the exterior perimeter of the recess comprises a rounded or beveled edge. In some embodiments of the electronic docking system, the electronic device comprises laptop computers, notebook computers, sub-notebook computers, netbook computers, netpad computers, media-streaming devices, handheld computers, Internet appliances, mobile smartphones, tablet computers, disposable tablet computers, personal digital assistants, and video game consoles. Suitable tablet computers include those with booklet, slate, and convertible configurations, known to those of skill in the art. In some embodiments, the electronic docking system further comprises: a second protrusion extending in a substantially perpendicular direction from the recess bottom face of the dock and a second alignment slot on the electronic device configured to accept the second protrusion. In some embodiments, the electronic docking system further comprises at least one of one or more magnets within the recess and one or more magnets inside or coupled with the electronic device and configured to mate with the one or more magnets in the recess. In some embodiments, the electronic docking system, the electronic device comprises a first alignment slot configured to accept a protrusion extending from the recess bottom face. In some embodiments, the electronic docking system, the electronic device comprises a first and second alignment slot configured to accept a first and second protrusion extending from the recess bottom face. In some embodiments, the electronic docking system further comprises at least one of: an indicator light on the dock; a motion sensor on the dock and an ambient light sensor on the dock. In some embodiments, the electronic docking system further comprises at least one of: an indicator light on the electronic device; a motion sensor on the electronic device and an ambient light sensor on the electronic device. In some embodiments of the electronic docking system, the motion sensor is a combination sensor also configurable as an ambient light sensor. In some embodiments of the electronic docking system, the pogo pin connector comprises at least one of: two or more pogo pins; three or more pogo pins; four or more pogo pins; five or more pogo pins; six or more pogo pins; seven or more pogo pins; eight or more pogo pins; nine or more pogo pins and 10 or more pogo pins. In some embodiments of the electronic docking system, the electrical contact comprises at least one of: two or more pogo pin receptacles; three or more pogo pin receptacles; four or more pogo pin; five or more pogo pin receptacles; six or more pogo pin receptacles; seven or more pogo pin receptacles; eight or more pogo pin receptacles; nine or more pogo pin receptacles and 10 or more pogo pin receptacles. In some embodiments, the contact ends of the pogo pins are rounded. In some embodiments, the pogo pins are tapered. In some embodiments of the electronic docking system, the system further comprises at least one support protrusion on the dock. In some embodiments of the electronic docking system, the at least one support protrusion extends vertically from a perimeter edge of the recess of the dock. In some embodiments of the electronic docking system, the indicator light indicates a charging mode, when illuminated, corresponding to a transfer of energy from the pogo pin connector to the electronic device. In some embodiments of the electronic docking system, the indicator light is activated when the electronic device is inserted in the dock recess and the electrical contact of the electronic device comes in contact with the pogo pins of the pogo pin connector. In some embodiments of the electronic docking system, the indicator light is further activated or deactivated by at least one of: the motion sensor; the ambient light sensor; a combination sensor and insertion or removal of the electronic device into or from the recess and making or breaking contact with the pogo pin connector. In some embodiments of the electronic docking system, the at least one sidewall in the dock recess comprises two or more side walls. In some embodiments of the electronic docking system, the two or more side walls comprise four side walls. In some embodiments of the electronic docking system, the at least one sidewall is tapered away from at least one of the first protrusion and the pogo pin connector. In some embodiments of the electronic docking system, the at least one support protrusion is positioned at least: on a posterior aspect of the recess; on a lateral aspect of the recess, or on an anterior aspect of the recess. In some embodiments of the electronic docking system, the at least one support protrusion is tapered away from at least one of the first protrusion and the pogo pin connector. In some embodiments of the electronic docking system, the recess of the dock is configured to guide an insertion of an edge of the electronic device such that the electrical contact of the electronic device accurately aligns with the pogo pin connector of the dock. In some embodiments of the electronic docking system, the recess of the dock is configured to guide an insertion of an edge of the electronic device such that the alignment slot of the electronic device accurately aligns with a protrusion extending from the recess bottom face. In some embodiments of the electronic docking system, the recess of the dock and the at least one proximal protrusion are configured to guide an insertion of an edge of the electronic device such that the electrical contact of the electronic device accurately aligns with the pogo pin connector of the dock. In some embodiments of the electronic docking system, the recess of the dock and the at least one proximal protrusion are configured to guide an insertion of an edge of the electronic device such that a first alignment slot and a second alignment slot of the electronic device accurately aligns with a first protrusion and a second protrusion extending from the recess bottom face. In some embodiments of the electronic docking system, the recess of the dock is generally rectangular in shape. In some embodiments of the electronic docking system, at least the first protrusion is generally oblong in shape. In some embodiments of the electronic docking system, at least one of the first and second protrusion extending from the recess bottom face have tapered or rounded side walls. In some embodiments of the electronic docking system, the at least one support protrusion on the dock is configured to provide support to the electronic device when placed in the recess of the dock.

Provided herein is a docking station for an electronic system comprising: a recess having at least one vertically oriented side wall and a recess bottom face; a pogo pin connector extending from the recess bottom face; a first protrusion extending in a substantially perpendicular direction from the recess bottom face and at least one support protrusion configured to provide support to an electronic device when placed in the recess of the docking station.

In some embodiments, the docking station further comprises a second protrusion extending in a substantially perpendicular direction from the recess bottom face. In some embodiments, the docking station further comprises at least one magnet within the recess configured to mate with one or more corresponding magnets inside or coupled with the electronic device when the electronic device is placed in the recess. In some embodiments of the docking station, the electronic device comprises at least one of: laptop computers, notebook computers, sub-notebook computers, netbook computers, netpad computers, media-streaming devices, handheld computers, Internet appliances, mobile smartphones, tablet computers, disposable tablet computers, personal digital assistants, and video game consoles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art. In some embodiments, the docking station further comprises at least one of: an indicator light; a motion sensor and an ambient light sensor. In some embodiments of the docking station, the motion sensor is a combination sensor also configurable as an ambient light sensor. In some embodiments of the docking station, the indicator light is further activated or deactivated by at least one of: the motion sensor; the ambient light sensor; insertion or removal of an electronic device into or from the recess and making or breaking contact with the pogo pin connector. In some embodiments of the docking station, the indicator light is further activated or deactivated by the combination sensor. In some embodiments of the electronic docking system, the pogo pin connector comprises at least one of: two or more pogo pins; three or more pogo pins; four or more pogo pins; five or more pogo pins; six or more pogo pins; seven or more pogo pins; eight or more pogo pins; nine or more pogo pins and 10 or more pogo pins. In some embodiments of the electronic docking system, the pogo pin connector comprises two pogo pins; three pogo pins; four pogo pins; five pogo pins; six pogo pins; seven pogo pins; eight pogo pins; nine pogo pins and 10 pogo pins. In some embodiments of the electronic docking system, the electrical contact comprises at least one of: two or more pogo pin receptacles; three or more pogo pin receptacles; four or more pogo pin; five or more pogo pin receptacles; six or more pogo pin receptacles; seven or more pogo pin receptacles; eight or more pogo pin receptacles; nine or more pogo pin receptacles and 10 or more pogo pin receptacles. In some embodiments of the electronic docking system, the electrical contact comprises two pogo pin receptacles; three pogo pin receptacles; four pogo pin; five pogo pin receptacles; six pogo pin receptacles; seven pogo pin receptacles; eight pogo pin receptacles; nine pogo pin receptacles and 10 pogo pin receptacles. In some embodiments of the docking station, the contact ends of the pogo pins are rounded. In some embodiments of the docking station, the pogo pins are tapered. In some embodiments of the docking station, the at least one support protrusion extends vertically from a perimeter edge of the recess of the dock. In some embodiments of the docking station, the indicator light indicates a charging mode, when illuminated, corresponding to a transfer of energy from the pogo pin connector to the electronic device. In some embodiments of the docking station, the indicator light is activated when an electronic device is inserted in the docking station recess and an electrical contact of the electronic device comes in contact with the pogo pins of the pogo pin connector. In some embodiments of the docking station, the motion sensor is configurable to activate (turn on) or deactivate (turn off) the indicator light on the docking station when motion is detected in the surrounding environment, or conversely, when no motion is detected in the surrounding environment. In some embodiments of the docking station, the ambient light sensor is configurable to activate (turn on) or deactivate (turn off) the indicator light on the docking station when the ambient light in the surrounding environment reaches specified levels. In some embodiments of the docking station, the combination sensor is configurable to activate (turn on) or deactivate (turn off) the indicator light by either motion or ambient light levels in the surrounding environment. In some embodiments of the docking station, the at least one sidewall comprises two or more side walls. In some embodiments of the docking station, the at least two or more side walls comprise four side walls. In some embodiments of the docking station, the at least one side wall is tapered away from at least one of the first protrusion and the pogo pin connector. In some embodiments of the docking station, the at least one support protrusion is positioned at least: on a posterior aspect of the recess; on a lateral aspect of the recess; or on an anterior aspect of the recess. In some embodiments of the docking station, the at least one support protrusion is tapered away from at least one of the first protrusion and the pogo pin connector. In some embodiments of the docking station, the recess of the docking station is configured to guide an insertion of an edge of an electronic device such that an electrical contact of the electronic device accurately aligns with the pogo pin connector of the dock. In some embodiments of the docking station, the recess of the docking station is configured to guide an insertion of an edge of an electronic device such that an alignment slot of an electronic device accurately aligns with a protrusion extending recess bottom face. In some embodiments of the docking station, the recess of the docking station and the at least one support protrusion are configured to guide an insertion of an edge of an electronic device such that the electrical contact of an electronic device accurately aligns with the pogo pin connector of the dock. In some embodiments of the docking station, the recess of the docking station and the at least one support protrusion are configured to guide an insertion of an edge of an electronic device such that an alignment slot of an electronic device accurately aligns with a proximal protrusion extending from the recess bottom face. In some embodiments of the docking station, the recess of the docking station is rectangular in shape. In some embodiments of the docking station, the at least one support protrusion on the dock is configured to provide support to an electronic device when placed in the recess of the dock.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a perspective view of the electronic device and docking station, showing our new design;

FIG. 2 is a front elevation view of the electronic device and docking station;

FIG. 3 is a rear elevation view of the electronic device and docking station;

FIG. 4 is a left side elevation view of the electronic device and docking station;

FIG. 5 is a right side elevation view of the electronic device and docking station;

FIG. 6 is a top plan view of the electronic device and docking station;

FIG. 7 is a bottom plan view of the electronic device and docking station;

FIG. 8 is a perspective of the docking station;

FIG. 9 is a front elevation view of the docking station;

FIG. 10 is a rear elevation view of the docking station;

FIG. 11 is a left side elevation view of the docking station;

FIG. 12 is a right side elevation view of the docking station;

FIG. 13 is a top plan view of the docking station;

FIG. 14 is a bottom plan view of the docking station;

FIG. 15 is a perspective of the electronic device;

FIG. 16 is a front elevation view of the electronic device;

FIG. 17 is a rear elevation view of the electronic device;

FIG. 18 is a left side elevation view of the electronic device;

FIG. 19 is a right side elevation view of the electronic device;

FIG. 20 is a top plan view of the electronic device; and

FIG. 21 is a bottom plan view of the electronic device.

The foregoing and other features of the present disclosure will become apparent from the following description and appended claims, taken in conjunction with the accompanying drawings. Understanding that these drawings depict only several embodiments in accordance with the disclosure and are therefore, not to be considered limiting of its scope, the disclosure will be described with additional specificity and detail through use of the accompanying drawings.

### DETAILED DESCRIPTION OF THE INVENTION

Described herein is an online-based story-sharing platform. This unique approach to Reminiscence Therapy allows users to record audio over photos as a way to share memories with family members who are suffering from a neurological or psychiatric condition. Similar to Reminiscence Therapy, this online-based story-sharing platform is a potential digital therapeutic that allows patients to reminisce about their past, but does not have the structured time requirement or one-on-one administration that is needed with these formal therapies. Furthermore, as an online-based story-sharing platform, it is readily accessible and can easily be used on an ongoing basis by patients.

The technology is unique in that it allows multiple family members, even if they are separated by time and place, to collaborate on the stories in just a few minutes a day. The platform transforms the short audio notes and individual photos into rich documentary-like stories that are then archived in a private and secured database. These stories can then be viewed easily with a tablet whenever the patient chooses and the interface is simple to operate. The tablet and docking station technology has the potential to be a practical and highly implementable adjunct behavioral intervention for a variety of patients, including those with dementia.

The online-based story-sharing platform is comprised of two components and is built on a proprietary delivery platform. In a preferred embodiment, the first component is the customized hardware tablet (the “Tablet”) that is specifically designed for use by individuals with neurodegenerative diseases or the elderly who are not able to operate standard consumer tablets. The Tablet does not display a user-interface for the elderly person. It presents itself more closely similar to a detachable photo frame that charges in a docking station without having to be plugged in. The absence of wires, buttons, or confusing interfaces is critical to making the Product accessible to this patient or demographic group. Additionally, the Tablet is wrapped in a protective foam casing which increases durability of the Tablet if it is dropped or thrown. The speakers and sound system have been optimized to maximize volume and clarity in comparison to off the shelf tablets. The included dock allows for charging of the Tablet in a cordless manner and powerful magnets make sure the Tablet lands securely in the dock.

The Tablet Software (the “Software”), which drives the use of the Tablet, combines a simplified viewing experience for the end user while simultaneously incorporating monitoring and tracking functionality for caregivers and administrators. The user interface is designed so the end user does not need to press any buttons. The user simply lifts the Tablet (off of the docking station) and it automatically begins to play calming, positive, and personalized media. This software uses proprietary emotional recognition software that learns what content has the greatest positive effect on the end user and relays the information to the Software's artificial intelligence system, which can focus its collection and organization of added media to the positive subject categories.

The emotional recognition and patient monitoring components of the product serve at least two purposes. Firstly: They encourage the family and caregivers to upload more content. By observing the biofeedback responses and seeing their loved one (grandma, grandpa, etc.) smile while viewing the content, the family and caregiver will be more likely to upload more content and be more engaged in the process. Secondly: The Product will automatically customize content based on the monitoring. The Product uses the AI to fine-tune content, identify the best content, and request this content from the family and caregivers. This enables optimization of the content being delivered.

The second component of the online-based story-sharing platform is the Software stack which is comprised of proprietary software functionality that includes an easy-to-use interface, which allows families, friends, caregivers, and administrators to customize and organize the content displayed to the end user—the individual who is experiencing “social isolation” or certain neurodegenerative diseases.

Primary interaction with the Product by content observers and contributors such as family members is primarily driven through text message communication. Each paying customer is given a private “concierge phone number” and any photos or videos that are sent to that number will appear on the end users tablet. In addition, the concierge phone number can be given to the paying customers' family and friends so they can also contribute content to the end user. The concierge phone number can also be added to a group text between loved ones who typically share photos and videos with each other and in this use case, through no additional effort, the content will also be shared with the end user.

In addition to text messaging, the Product has several additional channels of content collection. These include The Product “Family Mobile App” (iOS/Android), The Product “Family Web App” and email. The Product “Family App” is guided by an artificial intelligence (AI) system known as “Rachel” for the purpose of this disclosure. The AI system may have any name or no name, for the purposes of this disclosure. Any suitable name for this (AI) system may be selected as is known to those of skill in the art.

Rachel asks group members (family, friends, caregivers, and others) to add photos, videos and audio with the long-term goal of learning about what types of content is most helpful to the end user. Rachel maximizes the amount of content that is added by working with individual contributors, asking questions, leading the contributor through a process to develop additional media content to be presented to the Tablet user. Contributors can work with Rachel through the Family App on their own schedule, taking time when available to create new content, which may generally take approximately 5-10 minutes per “story” added. Rachel also allows users to contribute content via text message, email, or the Product website, as adjuncts to the Family App.

In addition, the underlying Delivery Platform upon which the Product is built can be modified and adapted to support people with additional clinical indications, such as traumatic brain injury (TBI) as well as other cognitive deficits and behavioral symptoms (e.g., depression, anxiety, and apathy), to name but a few non-limiting examples.

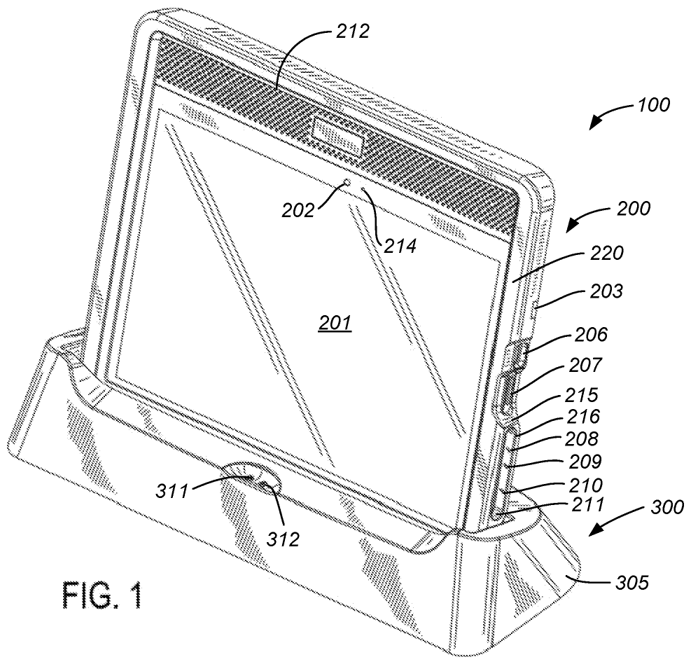

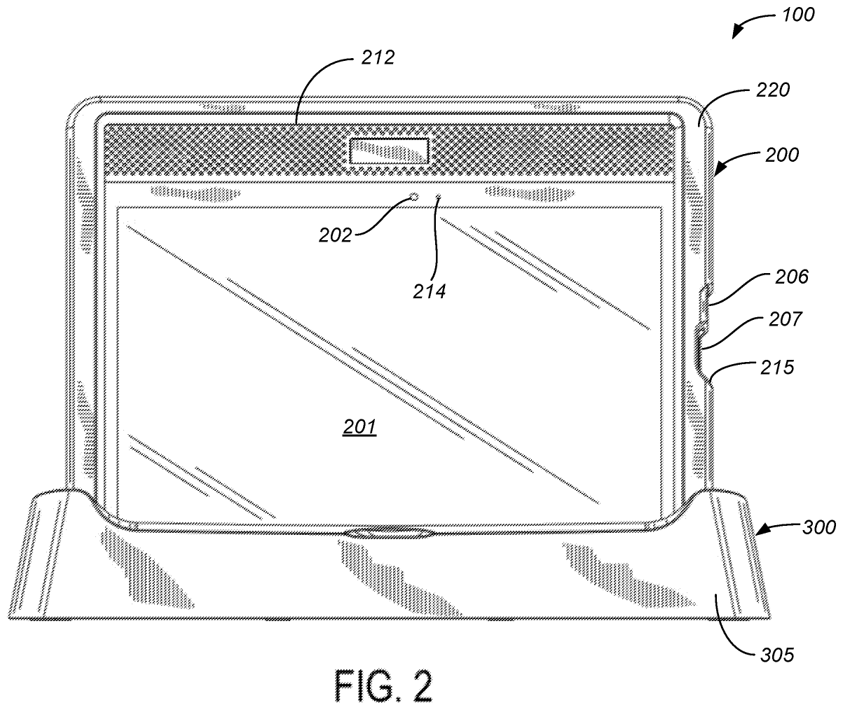

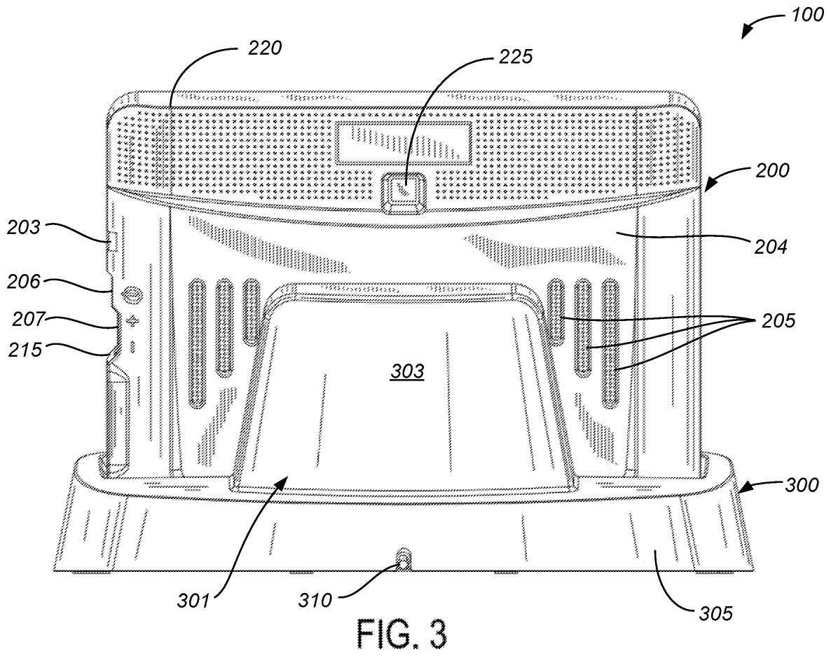

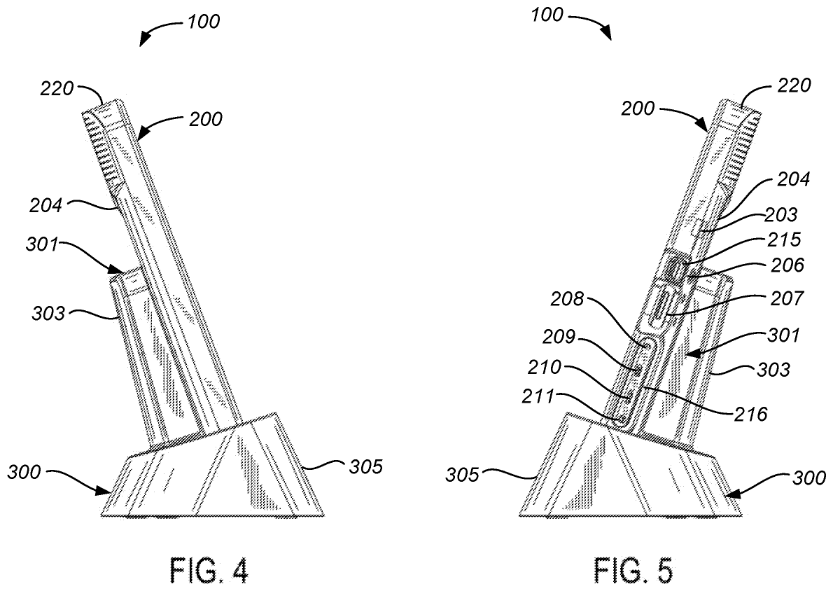

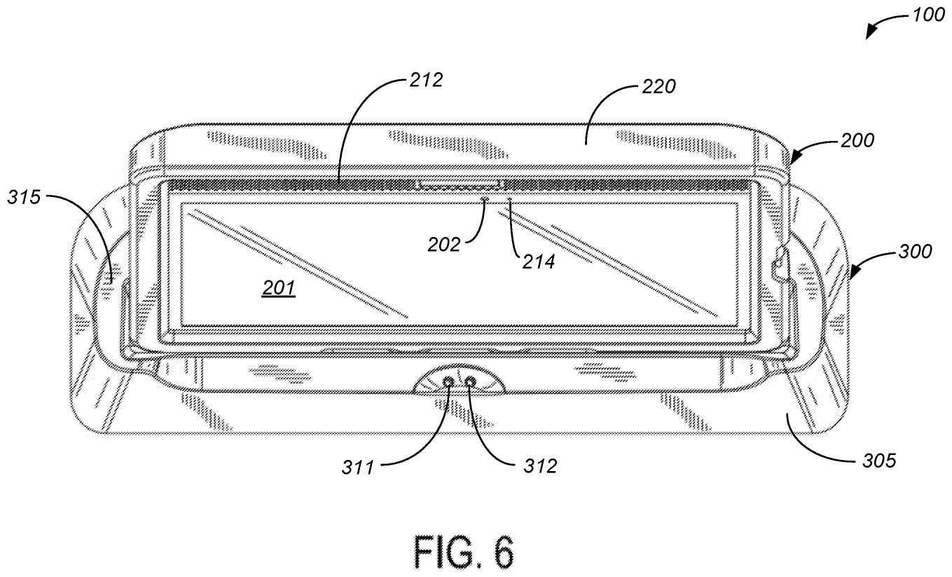

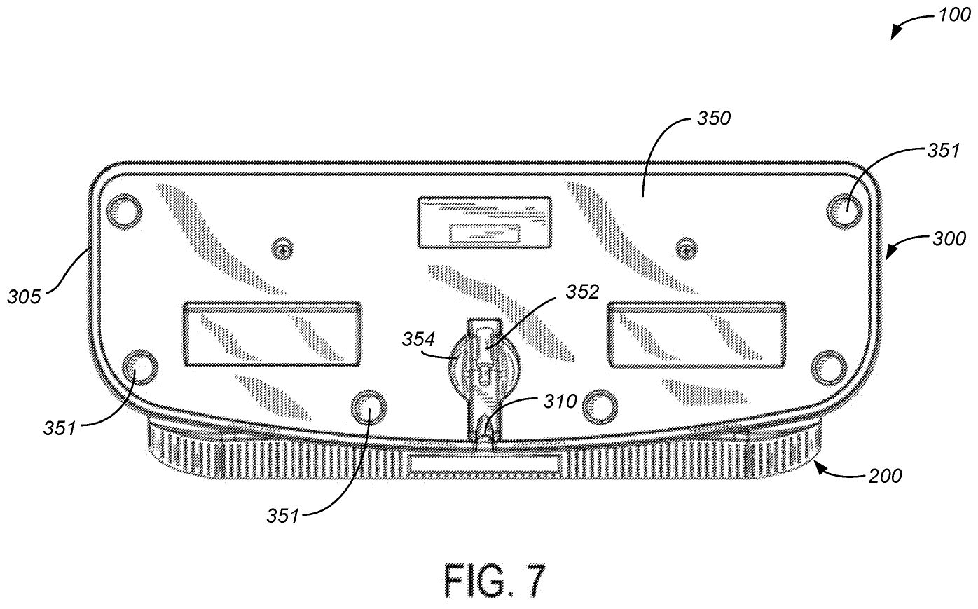

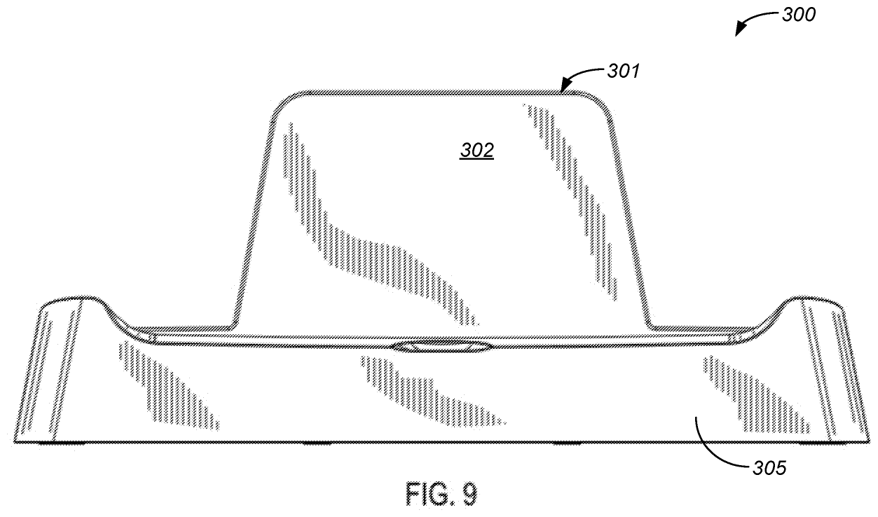

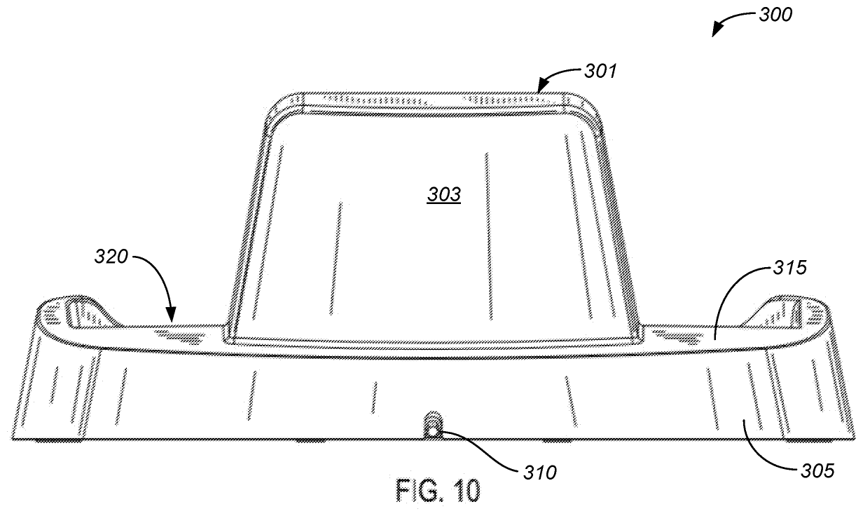

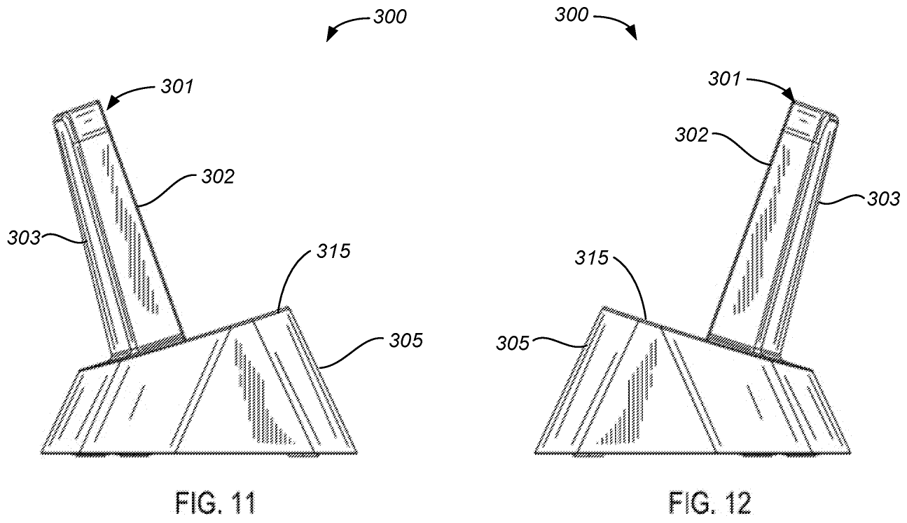

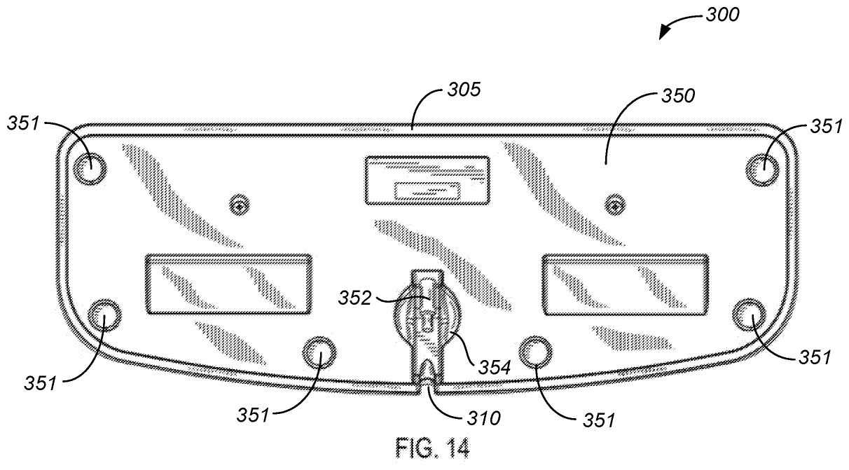

Provided herein is an electronic docking system 100. As illustrated in FIGS. 1-7, 8-14, the system comprises: a dock or docking station 300 comprising a recess 320 having at least one vertically oriented side wall 322 and a recess bottom face 321, wherein the dock comprises: a pogo pin connector 340 extending from the recess bottom face; a first protrusion 330 extending in a substantially perpendicular direction from the recess bottom face; and a second protrusion 330 extending in a substantially perpendicular direction from the recess bottom face. The electronic docking system 100 also comprises an electronic device, or more preferably an electronic tablet computer 200.

In some embodiments, each of the first and second protrusions 330 may further comprise an additional proximal protrusion 331 extending from each of the protrusions. In some embodiments, the first and second protrusions 330 and the proximal protrusion 331 are one in the same.

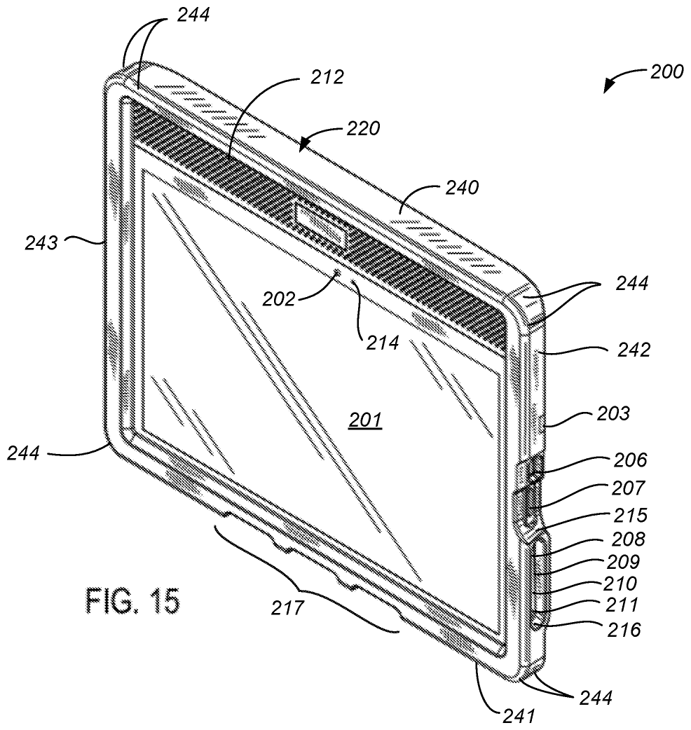

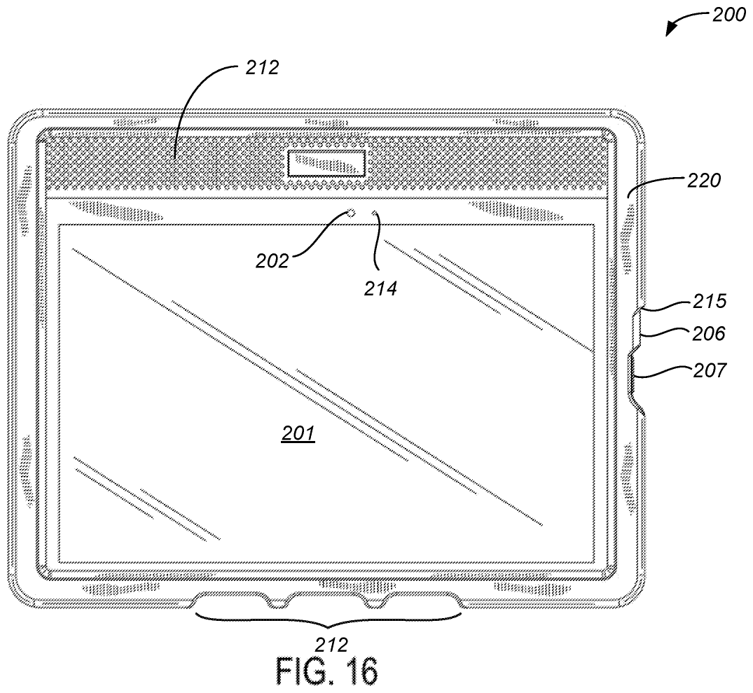

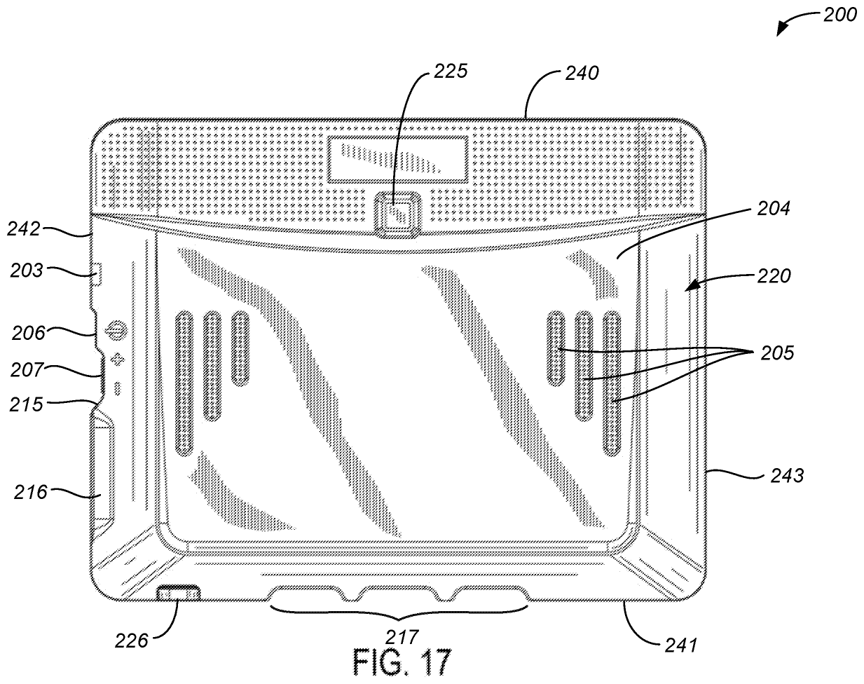

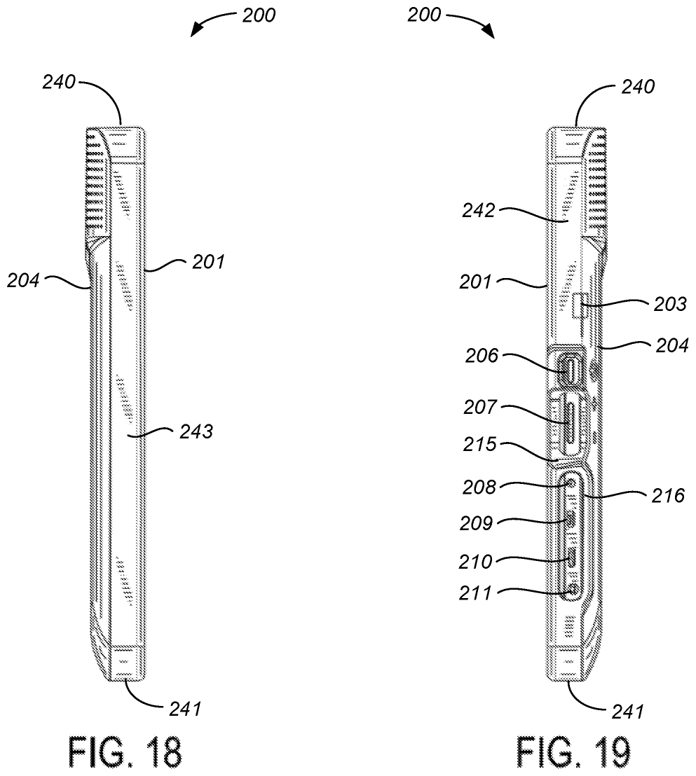

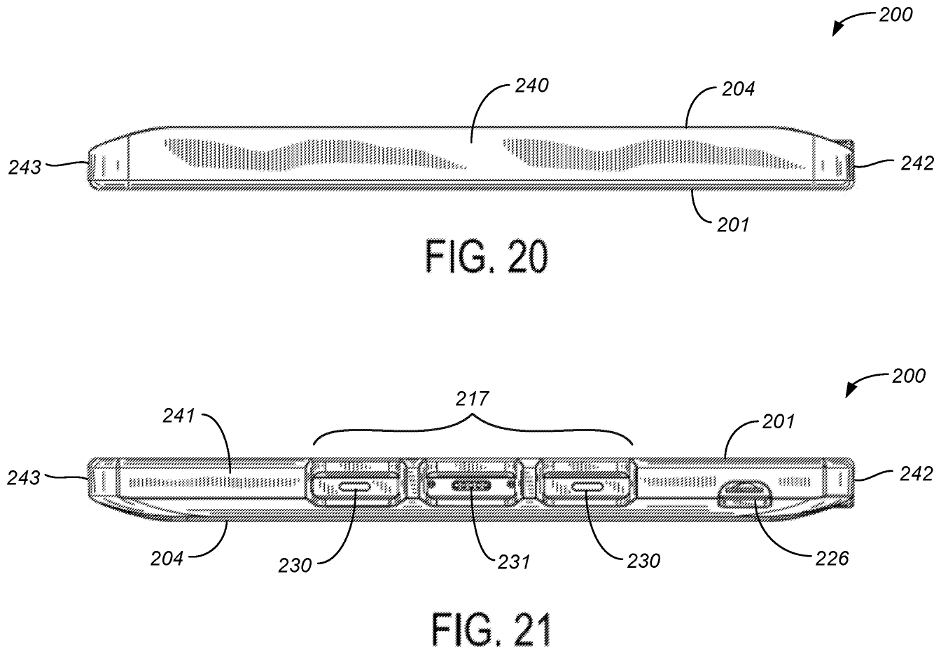

As further illustrated in FIGS. 15-21, the electronic device, or more preferably electronic tablet computer 200 comprises an electrical contact 231 configured to contact the pogo pin connector 340 in the recess 320 of the docking station 300; a first alignment slot 230 configured to accept the first protrusion 330 and/or proximal protrusion 331 extending from recess bottom face 321 of the docking station 300; and a second alignment slot 230 configured to accept the second protrusion 330 and/or proximal protrusion 331 extending from the recess bottom face 321 of the docking station 300.

In some embodiments of the electronic docking system 100, some embodiments of the docking station 300 further comprise at least one magnet (not shown) in the recess of the docking station. In some embodiments, the docking station comprises two magnets in the recess. In some embodiments, the docking station comprises three magnets in the recess. In some embodiments, the docking station comprises four magnets in the recess. In some embodiments, the at least one magnet is a permanent magnet. In some embodiments, the at least one magnet is a rare earth magnet. In some embodiments, the at least one magnet is a neodymium magnet. The at least one magnet enhances the docking action between the docking station and the electronic device. Preferably, the at least one magnet is strong enough to snap the electronic device in place in the recess once it is close enough to engage, but not so strong that it causes a user difficulty in removing the electronic device off of the docking station.

In some embodiments of the electronic docking system 100, some embodiments of the electronic device 200 further comprise at least one magnet (not shown) in the casing along the inferior edge that mates with the recess of the docking station. In some embodiments, the electronic device comprises two magnets in the casing along the inferior edge that mates with the recess of the docking station. In some embodiments, the electronic device comprises three magnets in the casing along the inferior edge that mates with the recess of the docking station. In some embodiments, the electronic device comprises four magnets in the casing along the inferior edge that mates with the recess of the docking station. In some embodiments, the at least one magnet is a permanent magnet. In some embodiments, the at least one magnet is a rare earth magnet. In some embodiments, the at least one magnet is a neodymium magnet. The at least one magnet enhances the docking action between the docking station and the electronic device. Preferably, the at least one magnet is strong enough to snap the electronic device in place in the recess once it is close enough to engage, but not so strong that it causes a user difficulty in removing the electronic device off of the docking station.

In some embodiments of the electronic docking system 100, at least a portion of the exterior perimeter of the recess 320 further comprises a rounded or beveled edge 323. The rounded or beveled edge 323 provides a smooth alignment transition surface when placing the tablet 200 into the docking station 300.

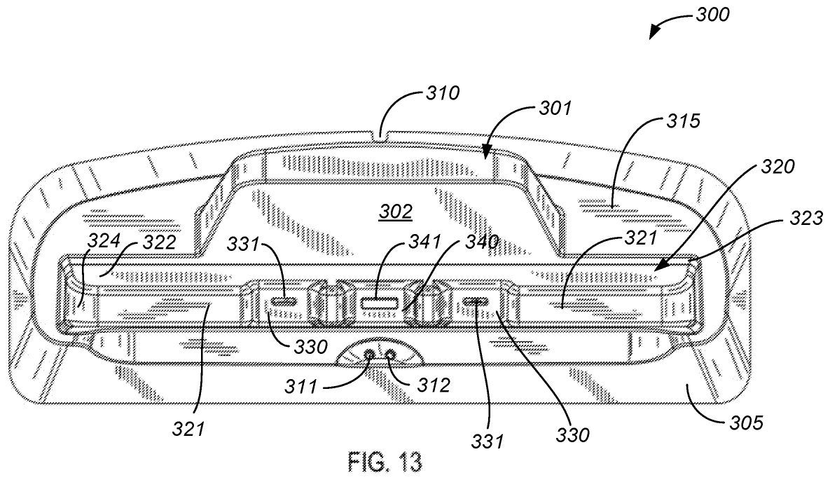

As clearly illustrated in FIGS. 1 and 13, some embodiments of the dock further comprise at least one of a charge indicator 311 and an ambient light sensor 312. In some embodiments of the dock further comprise at least one motion sensor (not shown). In some embodiments, the ambient light sensor can be either; a combination motion sensor and ambient light sensor (312/313) or may alternately be a separate sensor (not shown). As noted in the figures, the indicator 311, motion sensor, and ambient light sensor 312 would ideally be positioned on the docking station such that they are provided clear and unobstructed access to the surroundings for clear field of view and would be easily visible to a user. In a preferred embodiment, the indicator 311, motion sensor, and ambient light sensor 312 are positioned on an anterior or superior surface of the docking station such that they would not be obstructed or blocked by the electronic device when the electronic device was in the recess of the docking station.

In some embodiments the charge indicator 311 would be an LED light that would indicate that the tablet 200 is secured in the dock 300 and being charged. In some embodiments, the indicator 311 would be a multi-colored LED light configurable to distinguish changes in the charging level of the tablet 200. For example; red when it is less than fully charged, green when fully charged and even a third color such as yellow if the tablet is not in the dock or not properly seated within the dock.

In some embodiments, the electronic device 200 comprises laptop computers, notebook computers, sub-notebook computers, netbook computers, netpad computers, media streaming devices, handheld computers, Internet appliances, mobile smartphones, tablet computers, disposable tablet computers, personal digital assistants, and video game consoles. Suitable tablet computers include those with booklet, slate, and convertible configurations, known to those of skill in the art. As noted previously, for the purposes of this disclosure, a tablet computer was selected for illustration purposes.

In some embodiments of the electronic docking system 100, the pogo pin connector 340 comprises 5 pogo pins 341. In some embodiments of the electronic device 200, the electrical contact 231 configured to contact the pogo pin connector 340 in the recess 320 of the docking station 300, comprises 5 pogo pin receptacles 341.

In some embodiments of the electronic docking system 100, the pogo pin connector 340 comprises at least one of: two or more pogo pins 341; three or more pogo pins 341; four or more pogo pins 341; five or more pogo pins 341; six or more pogo pins 341; seven or more pogo pins 341; eight or more pogo pins 341; nine or more pogo pins 341 and 10 or more pogo pins 341.

In some embodiments of the electronic docking system 100, the electrical contact 231 comprise at least one of: two or more pogo pin receptacles; three or more pogo pin receptacles; four or more pogo pin; five or more pogo pin receptacles; six or more pogo pin receptacles; seven or more pogo pin receptacles; eight or more pogo pin receptacles; nine or more pogo pin receptacles and 10 or more pogo pin receptacles.

In some embodiments, the contact ends of the pogo pins 341 are rounded. In some embodiments, the pogo pins 341 are tapered. In some embodiments, the contact ends of the pogo pins 341 are spring-loaded.

In some embodiments of the electronic docking system 100, the pogo pins 341 in the pogo pin connector 340 are spring loaded and comprise a travel range between about of 2.0 mm and about 5.0 mm.

In some embodiments of the electronic docking system 100, the pogo pins 341 in the pogo pin connector 340 are between about 3.5 mm long and about 8.5 mm long.

In some embodiments of the electronic docking system 100, the pogo pins 341 in the pogo pin connector 340 are between about 0.5 mm in diameter and about 2.0 mm in diameter.

In some embodiments of the electronic docking system 100, the pogo pins 341 in the pogo pin connector 340 are space apart from each other between about 1.5 mm and about 3.5 mm.

Those of skill in the art will recognize that there are many other optional configurations for providing the connectivity provided by the pogo pins 341 which would also be suitable for an alternate configuration of the electronic docking system 100.

In some embodiments of the electronic docking system 100, the indicator 321 is a light or an LED that indicates a charging mode; which, when illuminated, corresponds to a transfer of energy from the pogo pin connector 340 to the electrical contact 231 the electronic device.

In some embodiments of the electronic docking system 100, the indicator light 321 is activated when the electronic device 200 is inserted in the dock recess 320 and the electrical contact 321 of the electronic device comes in contact with the pogo pins 341 of the pogo pin connector 340.

In some embodiments of the electronic docking system, the indicator light is further activated or deactivated by at least one of: the motion sensor 311; the ambient light sensor 312; or a combination sensor. In some embodiments of the electronic docking system 100, the indicator light 311 is further activated or deactivated by insertion or removal of the electronic device 200 into or from the recess 320 of the dock 300 by making or breaking contact between the pogo pin connector 340 and the electrical contact 231.

In some embodiments of the electronic docking system, the electronic device, or more broadly the digital processing device 200 is configured with a display 201. In any one of the embodiments, the digital processing device includes a display to send visual information to a user. Visual information may include pictures, videos, etc. In some embodiments, the display is a cathode ray tube (CRT). In some embodiments, the display is a liquid crystal display (LCD). In some embodiments, the display is a thin film transistor liquid crystal display (TFT-LCD). In some embodiments, the display is an organic light emitting diode (OLED) display. In various some embodiments, on OLED display is a passive-matrix OLED (PMOLED) or active-matrix OLED (AMOLED) display. In some embodiments, the display is a plasma display. In some embodiments, the display is a video projector. In still some embodiments, the display is a combination of devices such as those disclosed herein.

In some embodiments of the electronic docking system 100, the electronic device 200, is further equipped with a camera 202 to send visual information to a recipient that is not the user, such as a family member, caregiver or healthcare provider. Such information may include visual reactions when the user sees pictures or hears sounds displayed on the electronic device.

In some embodiments of the electronic docking system 100, the electronic device 200, is further equipped with an auxiliary camera 225 for reading machine readable code, or “Quick Response” code (i.e.: QR code) or matrix barcodes, (two-dimensional) barcodes. This type of coding in general is a machine-readable optical label that contains information about the item to which it is attached. A QR code uses four standardized encoding modes (numeric, alphanumeric, byte/binary, and kanji) to efficiently store data; extensions may also be used. Applications commonly include product tracking, item identification, time tracking, document management, and general marketing. A QR code consists of black squares arranged in a square grid on a white background, which can be read by an imaging device such as a camera, and processed using Reed-Solomon error correction until the image can be appropriately interpreted. The required data is then extracted from patterns that are present in both horizontal and vertical components of the image. QR codes are now used in a much broader context, including both commercial tracking applications and convenience-oriented applications aimed at mobile-phone users (termed mobile tagging). QR codes may be used to display text to the user, to add a vCard contact to the user's device, to open a Uniform Resource Identifier (URI), or to compose an email or text message.

In some embodiments of the electronic docking system 100, the electronic device 200, is further equipped with a microphone (203) to send auditory information to a recipient that is not the user, such as a family member, caregiver or healthcare provider. Such information may include laughter, crying or spoken words uttered by the user.

In some embodiments of the electronic docking system 100, the electronic device 200, is further equipped with a power indicator 214 to let the user know that the electronic device is powered “on” or “off”. Such an indicator is often displayed above the monitor screen, near the power switch, or another convenient location easily visible to the user. In many types of electronic devices comprising built-in keyboards or mouse pads, such an indicator may be displayed near those components.

In some embodiments of the electronic docking system 100, the electronic device 200, is further equipped with one or more speakers 212 to send auditory information to the user, such as music, sounds, spoken words. In some embodiments the speaker or speakers 212 are configured for high volume to aid a hearing impaired user. In some embodiments, the electronic device 200, is equipped with two speakers 212. In some embodiments, the electronic device 200, is equipped with three speakers 212. In some embodiments, the electronic device 200, is equipped with four speakers 212. In some embodiments of the electronic docking system 100, the electronic device 200, is further equipped with headphone jack 211 for wired headphones or Bluetooth® compatible software and hardware for wireless headphones. In some embodiments of the electronic docking system 100, the electronic device 200, is further equipped with adapters for a Micro-HDMI Plug 209 and a Micro-USB Plug 210. In some embodiments of the electronic docking system 100, the electronic device 200, is further equipped with a Power Button 206, a Volume Control 207 and a Power Plug 208.

In some embodiments, the electronic device 200, is further equipped with a durable casing 220, having a front 201 and a back 205, a superior edge 240, an inferior edge 241, a right edge 242 and a left edge 243, configurable to withstand unintentional drops or mishandling. In any one of the embodiments, the durable casing 220 is also configured with rounded edges and corners 244.

In some embodiments the durable casing 220 is configured with intentionally large rounded edges 244, roughened surfaces and other features, such as indents or handles to facilitate easy handling and solid gripping by an infirmed user.

In some embodiments the durable casing 220 is configured with vents 205 for heat dissipation.

In some embodiments the durable casing 220 is configured with indented recesses or connector relief areas 215, 216, 217 to securely house and protect adapters for auxiliary connectors.

In some embodiments of the electronic docking system, the electronic device, or more broadly the digital processing device 200 is configured with a processor and memory (not shown) that is configurable for remote updates through the docking station 300.

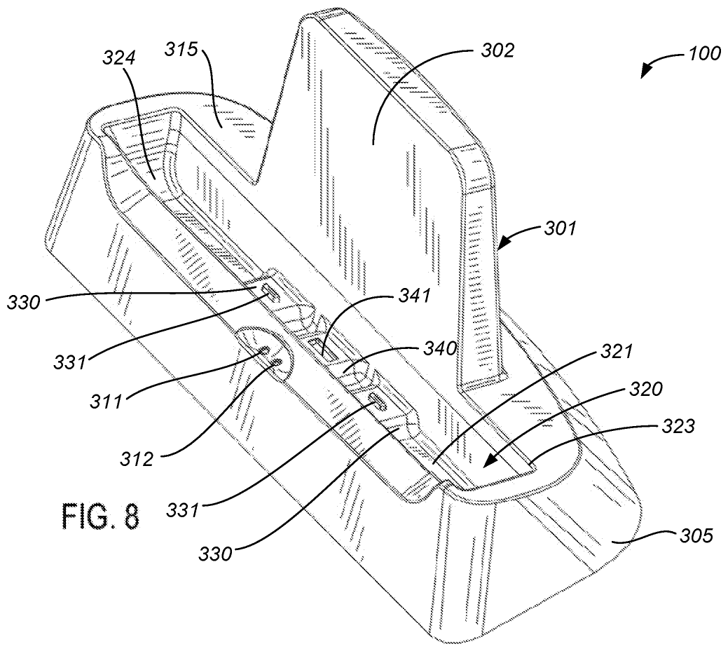

Referring once again to FIGS. 8-14, the dock or docking station 300 comprising a recess 320 having at least one vertically oriented side wall 322 and a recess bottom face 321, wherein the dock recess 320 further comprises: a pogo pin connector 340 extending from the recess bottom face; one or more protrusions 330 extending in a substantially perpendicular direction from the recess bottom face;

In some embodiments of the dock or docking station 300, each protrusion 330 further comprises a proximal protrusion 331 extending from the proximal surface of the protrusions. In some embodiments, the first and second protrusions 330 and the proximal protrusion 331 are one in the same.

In some embodiments, the docking station recess 320 is configured with between at least one vertically oriented side wall 322 and four vertically oriented side walls 322. The superior edge of each vertically oriented side wall 322 may further comprise a rounded, chamfered or beveled edge 323. Further still, the inferior edge of each vertically oriented side wall 322 may further comprise a rounded, chamfered or beveled inferior edge 324.

In some embodiments, the recess 320 in the dock 300 is generally rectangular in shape. Although one of skill in the art would recognize that the recess 320 could be virtually any shape that would accommodate the mating electronic device it was intended to support.

In some embodiments, the at least one vertically oriented side wall 322 in the recess 320 of the dock 300 is tapered away from at least one of the first protrusion 340 and/or the second protrusion 340.

In some embodiments of the electronic docking system 100, the dock further comprises at least one support protrusion 301 extending vertically from a perimeter edge of the recess vertically oriented side wall 322. The at least one support protrusion 301 may be vertically oriented at any orientation between about 45 degrees and 90 degrees (perpendicular) to a proximal surface 315 of the docking station 300.

In any embodiment comprising both a recess 320 with at least one vertically oriented side wall 322, and at least one support protrusion 301 extending vertically from a perimeter edge of the recess comprising the same vertically oriented side wall 322, it is anticipated that the at least one anterior surface 302 of the support protrusion 301 and at least one corresponding vertically oriented side wall 322 would at least be parallel, or even in the same plane. A posterior surface 303 of the at least one support protrusion 301 may or may not be parallel to the anterior face.

Alternatively, one or more of the at least one support protrusion 301 extending vertically from a perimeter edge of the recess 320 may be positioned at an angle that is at an angle slightly different or more obtuse than that of one or more of the vertically oriented side walls 322, to act as a tapered guide to the electronic device 200 for ease of placement in the recess 320.

In some embodiments, the recess 320 of the dock is configured to guide an insertion of an edge (i.e.: 241, 242, 243) of the electronic device 200 such that the electrical contact 231 of the electronic device accurately aligns with the pogo pin connector 340 of the dock 300.

In some embodiments, the recess 320 of the dock is configured to guide an insertion of an edge (i.e.: 241, 242, 243) of the electronic device 200 such that the alignment slot(s) 230 of the electronic device 200 accurately aligns with the protrusion(s) 330 extending from the recess bottom face 321.

In some embodiments, the recess 320 of the dock combined with the at least one support protrusion 301 extending generally vertically from a perimeter edge of the recess is configured to guide an insertion of an edge (i.e.: 241, 242, 243) of the electronic device 200 such that the electrical contact 231 of the electronic device accurately aligns with the pogo pin connector 340 of the dock 300.

In some embodiments, the recess 320 of the dock combined with the at least one support protrusion 301 extending generally vertically from a perimeter edge of the recess is configured to guide an insertion of an edge (i.e.: 241, 242, 243) of the electronic device 200 such that the alignment slot(s) 230 of the electronic device 200 accurately aligns with the protrusion(s) 330 extending from the recess bottom face 321.

In some embodiments, at least one protrusion(s) 330 is oblong in shape.

In some embodiments, at least one proximal protrusion(s) 331 is oblong in shape.

In some embodiments, at least one proximal protrusion(s) 331 is cylindrical in shape.

In some embodiments of the dock 300, the base 305 of the dock may be oblong in shape.

In some embodiments of the dock 300, the base 305 of the dock may be weighted down to prevent accidental tipping or unintentional movement by the user.

In some embodiments of the dock 300, the inferior surface 350 of the base 305 of the dock may be equipped with an inferior 354 and or posterior access port 310 configured for an external power connection 352. Further, the base 305 of the dock may be equipped with a plurality of feet, such as rubber pads 351 or an equivalent structure to protect a furniture surface or minimize noises when the base is moved or slid across a surface.

As used herein, and unless otherwise specified, the term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 30%, 25%, 20%, 15%, 10%, 9%, 8%, 7%, 6%, 5%, 4%, 3%, 2%, 1%, 0.5%, 0.1%, or 0.05% of a given value or range. In certain embodiments, the term “about” or “approximately” means within 40.0 mm, 30.0 mm, 20.0 mm, 10.0mm 5.0 mm 1.0 mm, 0.9 mm, 0.8 mm, 0.7 mm, 0.6 mm, 0.5 mm, 0.4 mm, 0.3 mm, 0.2 mm or 0.1 mm of a given value or range. In certain embodiments, the term “about” or “approximately” means within 5.0 kg, 2.5 kg, 1.0 kg, 0.9 kg, 0.8 kg, 0.7 kg, 0.6 kg, 0.5 kg, 0.4 kg, 0.3 kg, 0.2 kg or 0.1 kg of a given value or range, including increments therein. In certain embodiments, the term “about” or “approximately” means within 1 hour, within 45 minutes, within 30 minutes, within 25 minutes, within 20 minutes, within 15 minutes, within 10 minutes, within 5 minutes, within 4 minutes, within 3 minutes within 2 minutes, or within 1 minute. In certain embodiments, the term “about” or “approximately” means within 20.0 degrees, 15.0 degrees, 10.0 degrees, 9.0 degrees, 8.0 degrees, 7.0 degrees, 6.0 degrees, 5.0 degrees, 4.0 degrees, 3.0 degrees, 2.0 degrees, 1.0 degrees, 0.9 degrees, 0.8 degrees, 0.7 degrees, 0.6 degrees, 0.5 degrees, 0.4 degrees, 0.3 degrees, 0.2 degrees, 0.1 degrees, 0.09 degrees. 0.08 degrees, 0.07 degrees, 0.06 degrees, 0.05 degrees, 0.04 degrees, 0.03 degrees, 0.02 degrees or 0.01 degrees of a given value or range, including increments therein.

As used herein, and unless otherwise specified, the term “plurality”, and like terms, refers to a number (of things) comprising at least one (thing), or greater than one (thing), as in “two or more” (things), “three or more” (things), “four or more” (things), etc.

As used herein, the terms “connected”, “operationally connected”, “coupled”, “operationally coupled”, “operationally linked”, “operably connected”, “operably coupled”, “operably linked,” and like terms, refer to a relationship (mechanical, linkage, coupling, etc.) between elements whereby operation of one element results in a corresponding, following, or simultaneous operation or actuation of a second element. It is noted that in using said terms to describe inventive embodiments, specific structures or mechanisms that link or couple the elements are typically described. However, unless otherwise specifically stated, when one of said terms is used, the term indicates that the actual linkage or coupling may take a variety of forms, which in certain instances will be readily apparent to a person of ordinary skill in the relevant technology.

As used herein, the terms “comprises”, “comprising”, or any other variation thereof, are intended to cover a nonexclusive inclusion, such that a process, method, article, or apparatus that comprises a list of elements does not include only those elements but may include other elements not expressly listed or inherent to such process, method, article, or apparatus.

As used herein, the terms “user”, “subject” or “patient” are used interchangeably. As used herein, the terms “subject” and “subjects” refers to an animal (e.g., birds, reptiles, and mammals), preferably a mammal including a primate (e.g., a monkey, chimpanzee, and a human). In certain embodiments, the mammal is 0 to 6 months old, 6 to 12 months old, 1 to 5 years old, 5 to 10 years old, 10 to 15 years old, 15 to 20 years old, 20 to 25 years old, 25 to 30 years old, 30 to 35 years old, 35 to 40 years old, 40 to 45 years old, 45 to 50 years old, 50 to 55 years old, 55 to 60 years old, 60 to 65 years old, 65 to 70 years old, 70 to 75 years old, 75 to 80 years old, 80 to 85 years old, 85 to 90 years old, 90 to 95 years old, 95 to 100 years old or over 100 years old. In a preferred embodiment, the subject or patient is a human. In certain embodiments, the human is 40 to 60 years old, 60 to 70 years old, 70 to 80 years old, 80 to 90 years old, or 90 to 110 years old. The upper limit of the natural lifespan or maximum lifespan of a human is generally accepted to be about 125 years.

As used herein, and unless otherwise specified, the term “vertically oriented” and similar terms mean; generally perpendicular to, at, or near, right angles to a horizontal plane; in a direction, or having an alignment such that the top of a thing is above the bottom. In certain embodiments, the term “vertically oriented” means within ±20.0 degrees, ±15.0 degrees, ±10.0 degrees, ±9.0 degrees, ±8.0 degrees, ±7.0 degrees, ±6.0 degrees, ±5.0 degrees, ±4.0 degrees, ±3.0 degrees, ±2.0 degrees, ±1.0 degrees, ±0.9 degrees, ±0.8 degrees, ±0.7 degrees, ±0.6 degrees, ±0.5 degrees, ±0.4 degrees, ±0.3 degrees, ±0.2 degrees or ±0.1 degrees of a given value or range, including increments therein.

As used herein, and unless otherwise specified, the term “horizontally oriented” and similar terms mean; generally perpendicular to, at, or near, right angles to a vertical plane; in a direction, or having an alignment such that the top of a thing is generally on, or near the same plane as the bottom, both being parallel or near parallel to the horizon. In certain embodiments, the term “horizontally oriented” means within ±20.0 degrees, ±15.0 degrees, ±10.0 degrees, ±9.0 degrees, ±8.0 degrees, ±7.0 degrees, ±6.0 degrees, ±5.0 degrees, ±4.0 degrees, ±3.0 degrees, ±2.0 degrees, ±1.0 degrees, ±0.9 degrees, ±0.8 degrees, ±0.7 degrees, ±0.6 degrees, ±0.5 degrees, ±0.4 degrees, ±0.3 degrees, ±0.2 degrees or ±0.1 degrees of a given value or range, including increments therein.

As used herein, and unless otherwise specified, the term “substantially perpendicular” and similar terms mean generally at or near 90 degrees to a given line, or surface or to the ground. In certain embodiments, the term “substantially perpendicular” means within ±20.0 degrees, ±15.0 degrees, ±10.0 degrees, ±9.0 degrees, ±8.0 degrees, ±7.0 degrees, ±6.0 degrees, ±5.0 degrees, ±4.0 degrees, ±3.0 degrees, ±2.0 degrees, ±1.0 degrees, ±0.9 degrees, ±0.8 degrees, ±0.7 degrees, ±0.6 degrees, ±0.5 degrees, ±0.4 degrees, ±0.3 degrees, ±0.2 degrees or ±0.1 degrees of a given value or range, including increments therein.

As used herein, and unless otherwise specified, the term “generally rectangular” and similar terms mean comprising four sides, edges, surfaces or faces, placed or having parts placed at, or near right angles. In some embodiments, the term “generally rectangular” can refer to a parallelogram, where opposite sides are parallel and typically equal in length, wherein opposite angles are equal (angles “a” are the same, and angles “b” are the same). As used herein: Squares, Rectangles and Rhombuses are all Parallelograms. In certain embodiments, the term “generally rectangular” means within ±20.0 degrees, ±15.0 degrees, ±10.0 degrees, ±9.0 degrees, ±8.0 degrees, ±7.0 degrees, ±6.0 degrees, ±5.0 degrees, ±4.0 degrees, ±3.0 degrees, ±2.0 degrees, ±1.0 degrees, ±0.9 degrees, ±0.8 degrees, ±0.7 degrees, ±0.6 degrees, ±0.5 degrees, ±0.4 degrees, ±0.3 degrees, ±0.2 degrees or ±0.1 degrees of a given value or range, including increments therein.

As used herein, and unless otherwise specified, the term “generally oblong” and similar terms mean having a shape that resembles an elongated rectangle or oval shape, wherein the shape is longer than it is wide in one dimension.

As used herein, and unless otherwise specified, the terms “dock”, “docking station” and similar terms mean means a base or porting device in which an electronic device such as a laptop computer, smartphone, or other mobile device may be placed for charging, for digital exchange of instructions, providing access to a power supply and to peripheral devices or auxiliary features.

As used herein, and unless otherwise specified, the term “Product” means or refers to the entire system, the docking station, the electronic device, the software or any Apps (or applications) developed for use with the entire system or any of its components.

In some embodiments, the platforms, media, methods, and applications described herein include a digital processing device, a processor, or use of the same. In some embodiments, the digital processing device includes one or more hardware central processing units (CPU) that carry out the device's functions. In still some embodiments, the digital processing device comprises an operating system configured to perform executable instructions. In some embodiments, the digital processing device is optionally connected to a computer network. In some embodiments, the digital processing device is optionally connected to the Internet such that it accesses the World Wide Web. In still some embodiments, the digital processing device is optionally connected to a cloud computing infrastructure. In some embodiments, the digital processing device is optionally connected to an intranet. In some embodiments, the digital processing device is optionally connected to a data storage device.

In accordance with the description herein, suitable digital processing devices include, by way of non-limiting examples, server computers, desktop computers, laptop computers, notebook computers, sub-notebook computers, netbook computers, netpad computers, set-top computers, handheld computers, Internet appliances, mobile smartphones, tablet computers, disposable tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, the digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®.

In some embodiments, the device includes a storage and/or memory device. The storage and/or memory device is one or more physical apparatus used to store data or programs on a temporary or permanent basis. In some embodiments, the device is volatile memory and requires power to maintain stored information. In some embodiments, the device is non-volatile memory and retains stored information when the digital processing device is not powered. In some embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In some embodiments, the device is a storage device including, by way of non-limiting examples, CD-ROMs, DVDs, flash memory devices, magnetic disk drives, magnetic tapes drives, optical disk drives, and cloud computing based storage. In some embodiments, the storage and/or memory device is a combination of devices such as those disclosed herein.

In some embodiments, the digital processing device includes a display to send visual information to a user. In some embodiments, the display is a cathode ray tube (CRT). In some embodiments, the display is a liquid crystal display (LCD). In some embodiments, the display is a thin film transistor liquid crystal display (TFT-LCD). In some embodiments, the display is an organic light emitting diode (OLED) display. In various some embodiments, on OLED display is a passive-matrix OLED (PMOLED) or active-matrix OLED (AMOLED) display. In some embodiments, the display is a plasma display. In some embodiments, the display is a video projector. In still some embodiments, the display is a combination of devices such as those disclosed herein.

The tablet and docking station technology solution described herein is unique among digital reminiscence therapy platforms in that personalizes RT for the patient into custom stories, and can optimize content being delivered to the patient using machine learning, proprietary emotional recognition software, as well as feedback from the family. This level of technology allows for an automatic process for personalized therapy, optimizing potential response from patients. Further, it utilizes and encourages family engagement, providing benefit to the caregiver as well as the patient. In addition, the solution is a truly scalable solution, in that it is a low-cost platform only relying on a custom tablet, family members' existing mobile devices, and the cloud. Therefore, this technology can be used at any home or senior care/memory care facility, and in rural and isolated settings where the purchase of large shared kiosks or VR equipment, or specialized staff or caregiver training, may not be practical. Due to its simple user interface, a patient's openness to new technology is also not a concern (as it is with many other solutions). Patients are given a tablet with no menus or buttons; a patient simply picks up the tablet to begin viewing stories. Overcoming the possible fear of new technology by a patient is almost entirely circumvented.

The system features three key components: (1) an app that can be downloaded onto one or more family members' phones; (2) a cloud-based server; and (3) a tablet with a docking station that is used by the patient. The purpose of the system is to move stories from the family to the patient. The stories are moved through the cloud-based server, which runs on a secure cloud service platform, offering compute power, database storage, content delivery and other functionality primarily for businesses. The responsibility of the family app and the server together is to request story info via a custom AI chatbot—the AI chatbot chats with family members and directs them to upload photos and tell audio stories. Requested photos are based on themes such as weddings, vacations and birthdays, and when recording audio, the user has the option to speak freely or use prewritten scripts to help shape the story.

The online-based story-sharing platform, systems, and methods herein allow users to record audio or submit written descriptions or comments related to photos or videos to easily share memories with family members who are suffering from a neurological or psychiatric condition. As such, multiple family members from around the world and at different times may collaborate to form meaningful and therapeutic the stories in just a few minutes a day. The platform transforms the media and descriptions received by family members into rich documentary-like stories that may be archived in a private and secured database. These stories can then be viewed easily through a simple user interface via any media device whenever the patient chooses. Thus, RT patients can reminisce about their past in a structured or unstructured time or facility, without the necessity for one-on-one administration.

One aspect provided herein is reminiscence therapy platform for a patient user comprising: a contributing user mobile processor configured to provide a first mobile application comprising: a prompt module receiving and displaying a request for a first media from a contributing user; a first media module receiving the first media from the contributing user; and a server processor configured to provide a server application comprising: a request module generating the request and submitting the request to the prompt module; and a chronicle module receiving the first media and generating a story based on the first media; and a patient user mobile processor configured to provide a second mobile application comprising: a communications module receiving the story; a media output module presenting the story to the patient user; a reaction module measuring a reaction of the patient user while media output module presents the story to a patient user; and a feedback module transmitting the reaction to the request module; wherein the request module generates a subsequent request based on the reaction.

In some embodiments at least one the first media and the story comprises a photograph, a video, an image, a gif, an emoji, a text, or any combination thereof. In some embodiments the request comprises a media type, a media theme, a media subject, a media color, a media date, a media duration, or any combination thereof. In some embodiments the prompt module displays the request for the first media via a screen, a speakerphone, a phone call, a text message, a push notification, an email, or any combination thereof. In some embodiments the reaction comprises an anger parameter, a contempt parameter, a fear parameter, a happiness parameter, a surprise parameter, a sadness parameter, a symmetry parameter, an eye quadrant, an eye fixation time, an eye fixation duration, a button press, or any combination thereof. In some embodiments the reaction does not comprise a button press. In some embodiments the reaction module measures the reaction of the patient user through a facial recognition process. In some embodiments the facial recognition process is configured for facial recognition of the patient user having an age of greater than about 50 years. In some embodiments the facial recognition process comprises a computer learning process.

In some embodiments the contributing user comprises a plurality of contributing users. In some embodiments the chronicle module generates the story by performing at least the following: performing a facial recognition on the first media; performing object recognition on the first media; determining a geographic location associated with the first media. In some embodiments the facial recognition process comprises age recognition, sex recognition, environment recognition, object recognition, or any combination thereof. In some embodiments the chronicle module further stores the story.

In some embodiments of the platform, the server application further comprises a database storing a plurality of the templates. In some embodiments the first mobile application further comprises a descriptor module notifying the contributing user to submit a second media based on the first media and a template. In some embodiments the second media is further based on the third media. In some embodiments the first mobile application further comprises a second media module receiving the second media from the contributing user. In some embodiments the chronicle module further generates the story based on the second media. In some embodiments the second media comprises a photograph, a video, an image, a gif, an emoji, a text, or any combination thereof. In some embodiments the server application further comprises a media agglomeration module determining and receiving a third media from a third media source. In some embodiments the third media source comprises a social media image, a social media text, a social media video, a public media image, a public media text, a public media video, or any combination thereof. In some embodiments the chronicle module further generates the story based on the third media. In some embodiments the media agglomeration module determines the third media by a computer learning process. In some embodiments the prompt module receives the request through a cellular network, a wireless network, a Bluetooth signal, a wired signal, or any combination thereof. In some embodiments the first media module receives the first media through a cellular network, a wireless network, a Bluetooth signal, a wired signal, or any combination thereof. In some embodiments the chronicle module receives the first media through a cellular network, a wireless network, a Bluetooth signal, a wired signal, or any combination thereof. In some embodiments the communication module receives the story through a cellular network, a wireless network, a Bluetooth signal, a wired signal, or any combination thereof. In some embodiments the feedback module transmits the reaction through a cellular network, a wireless network, a Bluetooth signal, a wired signal, or any combination thereof.

Provided herein is an electronic docking system comprising: a docking station or dock comprising a recess having at least one vertically oriented side wall and a recess bottom face, wherein the dock comprises: a pogo pin connector extending from the recess bottom face; a first protrusion extending in a substantially perpendicular direction from the recess bottom face; and an electronic device having a device width, a device thickness and a device length: an electrical contact configured to contact the pogo pin connector and a first alignment slot configured to accept the first protrusion, wherein at least a portion of an exterior perimeter of the recess comprises a rounded or beveled edge.

In some embodiments of the electronic docking system, the dock further comprises a first proximal protrusion extending from the first protrusion.

In some embodiments of the electronic docking system, the electronic device comprises laptop computers, notebook computers, sub-notebook computers, netbook computers, netpad computers, media-streaming devices, handheld computers, Internet appliances, mobile smartphones, tablet computers, disposable tablet computers, personal digital assistants, and video game consoles. Suitable tablet computers include those with booklet, slate, and convertible configurations, known to those of skill in the art.

In some embodiments, the electronic docking system further comprises a second protrusion extending in a substantially perpendicular direction from the recess bottom face of the dock and a second alignment slot on the electronic device configured to accept the second protrusion.

In some embodiments, the electronic docking system further comprises a second proximal protrusion extending from the second protrusion

In some embodiments, the electronic docking system, some embodiments of the docking station further comprise at least one magnet (not shown) in the recess of the docking station. In some embodiments, the docking station comprises two magnets in the recess. In some embodiments, the docking station comprises three magnets in the recess. In some embodiments, the docking station comprises four magnets in the recess. In some embodiments, the at least one magnet is a permanent magnet. In some embodiments, the at least one magnet is a rare earth magnet. In some embodiments, the at least one magnet is a neodymium magnet. The at least one magnet enhances the docking action between the docking station and the electronic device. Preferably, the at least one magnet is strong enough to snap the electronic device in place in the recess once it is close enough to engage, but not so strong that it causes a user difficulty in removing the electronic device off of the docking station.

In some embodiments of the electronic docking system, some embodiments of the electronic device further comprise at least one magnet (not shown) in the casing along the inferior edge that mates with the recess of the docking station. In some embodiments, the electronic device comprises two magnets in the casing along the inferior edge that mates with the recess of the docking station. In some embodiments, the electronic device comprises three magnets in the casing along the inferior edge that mates with the recess of the docking station. In some embodiments, the electronic device comprises four magnets in the casing along the inferior edge that mates with the recess of the docking station. In some embodiments, the at least one magnet is a permanent magnet. In some embodiments, the at least one magnet is a rare earth magnet. In some embodiments, the at least one magnet is a neodymium magnet. The at least one magnet enhances the docking action between the docking station and the electronic device. Preferably, the at least one magnet is strong enough to snap the electronic device in place in the recess once it is close enough to engage, but not so strong that it causes a user difficulty in removing the electronic device off of the docking station.

In some embodiments, the electronic docking system further comprises at least one of: an indicator light on the dock; a motion sensor on the dock and an ambient light sensor on the dock.

In some embodiments, the electronic docking system further comprises at least one of: an indicator light on the electronic device; a motion sensor on the electronic device and an ambient light sensor on the electronic device.

In some embodiments of the electronic docking system, the motion sensor is a combination sensor also configurable as an ambient light sensor.

In some embodiments of the electronic docking system, the pogo pin connector comprises at least one of: two or more pogo pins; three or more pogo pins; four or more pogo pins; five or more pogo pins; six or more pogo pins; seven or more pogo pins; eight or more pogo pins; nine or more pogo pins and 10 or more pogo pins.

In some embodiments of the electronic docking system, the electrical contact comprises at least one of: two or more pogo pin receptacles; three or more pogo pin receptacles; four or more pogo pin; five or more pogo pin receptacles; six or more pogo pin receptacles; seven or more pogo pin receptacles; eight or more pogo pin receptacles; nine or more pogo pin receptacles and 10 or more pogo pin receptacles.

In some embodiments, the contact ends of the pogo pins are rounded. In some embodiments, the pogo pins are tapered.

In some embodiments of the electronic docking system, the system further comprises at least one support protrusion on the dock.

In some embodiments of the electronic docking system, the at least one support protrusion extends vertically from a perimeter edge of the recess of the dock.

In some embodiments of the electronic docking system, the indicator light indicates a charging mode, when illuminated, corresponding to a transfer of energy from the pogo pin connector to the electronic device.

In some embodiments of the electronic docking system, the indicator light is activated when the electronic device is inserted in the dock recess and the electrical contact of the electronic device comes in contact with the pogo pins of the pogo pin connector.

In some embodiments of the electronic docking system, the indicator light is further activated or deactivated by at least one of: the motion sensor; the ambient light sensor; a combination sensor and insertion or removal of the electronic device into or from the recess and making or breaking contact with the pogo pin connector.

In some embodiments of the electronic docking system, the at least one sidewall in the dock recess comprises two or more side walls.

In some embodiments of the electronic docking system, the two or more side walls comprise four side walls.

In some embodiments of the electronic docking system, the at least one sidewall is tapered away from at least one of the first protrusion and the pogo pin connector.

In some embodiments of the electronic docking system, the at least one support protrusion is positioned at least: on a posterior aspect of the recess; on a lateral aspect of the recess, or on an anterior aspect of the recess.

In some embodiments of the electronic docking system, the at least one support protrusion is tapered away from at least one of the first protrusion and the pogo pin connector.

In some embodiments of the electronic docking system, the recess of the dock is configured to guide an insertion of an edge of the electronic device such that the electrical contact of the electronic device accurately aligns with the pogo pin connector of the dock.

In some embodiments of the electronic docking system, the recess of the dock is configured to guide an insertion of an edge of the electronic device such that the alignment slot of the electronic device accurately aligns with a proximal protrusion extending from the tapered protrusion.

In some embodiments of the electronic docking system, the recess of the dock and the at least one proximal protrusion are configured to guide an insertion of an edge of the electronic device such that the electrical contact of the electronic device accurately aligns with the pogo pin connector of the dock.

In some embodiments of the electronic docking system, the recess of the dock is generally rectangular in shape.

In some embodiments of the electronic docking system, the first protrusion is generally oblong in shape.

In some embodiments of the electronic docking system, the at least one support protrusion on the dock is configured to provide support to the electronic device when placed in the recess of the dock.

Provided herein is a docking station for an electronic system comprising: a recess having at least one vertically oriented side wall and a recess bottom face; a pogo pin connector extending from the recess bottom face; a first protrusion extending in a substantially perpendicular direction from the recess bottom face and at least one support protrusion configured to provide support to an electronic device when placed in the recess of the docking station.

In some embodiments, the docking station further comprises a second protrusion extending in a substantially perpendicular direction from the recess bottom face.

In some embodiments, the docking station further comprises at least one magnet (not shown) in the recess of the docking station configured to mate with corresponding magnets in the electronic device. In some embodiments, the docking station comprises two magnets in the recess. In some embodiments, the docking station comprises three magnets in the recess. In some embodiments, the docking station comprises four magnets in the recess. In some embodiments, the at least one magnet is a permanent magnet. In some embodiments, the at least one magnet is a rare earth magnet. In some embodiments, the at least one magnet is a neodymium magnet. The at least one magnet enhances the docking action between the docking station and the electronic device, which comprises one or more corresponding magnets configured to mate with the magnets in the recess of the docking station. Preferably, the at least one magnet is strong enough to snap the electronic device in place in the recess once it is close enough to engage, but not so strong that it causes a user difficulty in removing the electronic device off of the docking station.

In some embodiments of the docking station, the electronic device comprises at least one of: laptop computers, notebook computers, sub-notebook computers, netbook computers, netpad computers, media-streaming devices, handheld computers, Internet appliances, mobile smartphones, tablet computers, disposable tablet computers, personal digital assistants, and video game consoles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, the electronic device further comprises at least one magnet (not shown) in the casing along the inferior edge that mates with the recess of the docking station. In some embodiments, the electronic device comprises two magnets in the casing along the inferior edge that mates with the recess of the docking station. In some embodiments, the electronic device comprises three magnets in the casing along the inferior edge that mates with the recess of the docking station. In some embodiments, the electronic device comprises four magnets in the casing along the inferior edge that mates with the recess of the docking station. In some embodiments, the at least one magnet is a permanent magnet. In some embodiments, the at least one magnet is a rare earth magnet. In some embodiments, the at least one magnet is a neodymium magnet. The at least one magnet enhances the docking action between the docking station and the electronic device. Preferably, the at least one magnet is strong enough to snap the electronic device in place in the recess once it is close enough to engage, but not so strong that it causes a user difficulty in removing the electronic device off of the docking station.

In some embodiments, the docking station further comprises at least one of: an indicator light; a motion sensor and an ambient light sensor.

In some embodiments of the docking station, the motion sensor is a combination sensor also configurable as an ambient light sensor.

In some embodiments of the docking station, the indicator light is further activated or deactivated by at least one of: the motion sensor; the ambient light sensor; insertion or removal of an electronic device into or from the recess and making or breaking contact with the pogo pin connector.

In some embodiments of the docking station, the indicator light is further activated or deactivated by the combination sensor.

In some embodiments of the electronic docking system, the pogo pin connector comprises at least one of: two or more pogo pins; three or more pogo pins; four or more pogo pins; five or more pogo pins; six or more pogo pins; seven or more pogo pins; eight or more pogo pins; nine or more pogo pins and 10 or more pogo pins.

In some embodiments of the electronic docking system, the electrical contact comprises at least one of: two or more pogo pin receptacles; three or more pogo pin receptacles; four or more pogo pin; five or more pogo pin receptacles; six or more pogo pin receptacles; seven or more pogo pin receptacles; eight or more pogo pin receptacles; nine or more pogo pin receptacles and 10 or more pogo pin receptacles.

In some embodiments of the docking station, the contact ends of the pogo pins are rounded. In some embodiments of the docking station, the pogo pins are tapered.

In some embodiments of the docking station, the at least one support protrusion extends vertically from a perimeter edge of the recess of the dock.

In some embodiments of the docking station, the indicator light indicates a charging mode, when illuminated, corresponding to a transfer of energy from the pogo pin connector to the electronic device.

In some embodiments of the docking station, the indicator light is activated when an electronic device is inserted in the docking station recess and an electrical contact of the electronic device comes in contact with the pogo pins of the pogo pin connector.

In some embodiments of the docking station, the motion sensor is configurable to activate (turn on) or deactivate (turn off) the indicator light on the docking station when motion is detected in the surrounding environment, or conversely, when no motion is detected in the surrounding environment.

In some embodiments of the docking station, the ambient light sensor is configurable to activate (turn on) or deactivate (turn off) the indicator light on the docking station when the ambient light in the surrounding environment reaches specified levels.

In some embodiments of the docking station, the combination sensor is configurable to activate (turn on) or deactivate (turn off) the indicator light by either motion or ambient light levels in the surrounding environment.

In some embodiments of the docking station, the at least one sidewall comprises two or more side walls.

In some embodiments of the docking station, the at least two or more side walls comprise four side walls.

In some embodiments of the docking station, the at least one side wall is tapered away from at least one of the first protrusion and the pogo pin connector.

In some embodiments of the docking station, the at least one support protrusion is positioned at least: on a posterior aspect of the recess; on a lateral aspect of the recess; or on an anterior aspect of the recess.

In some embodiments of the docking station, the at least one support protrusion is tapered away from at least one of the first protrusion and the pogo pin connector.

In some embodiments of the docking station, the recess of the docking station is configured to guide an insertion of an edge of an electronic device such that an electrical contact of an electronic device accurately aligns with the pogo pin connector of the dock.

In some embodiments of the docking station, the recess of the docking station is configured to guide an insertion of an edge of an electronic device such that an alignment slot of an electronic device accurately aligns with a protrusion extending from the recess bottom face.

In some embodiments of the docking station, the recess of the docking station and the at least one support protrusion are configured to guide an insertion of an edge of an electronic device such that an electrical contact of an electronic device accurately aligns with the pogo pin connector of the dock.

In some embodiments of the docking station, the recess of the docking station and the at least one support protrusion are configured to guide an insertion of an edge of an electronic device such that an alignment slot of an electronic device accurately aligns with a protrusion extending from the recess bottom face.

In some embodiments of the docking station, the recess of the docking station is rectangular in shape.

In some embodiments of the docking station, the at least one support protrusion on the dock is configured to provide support to an electronic device when placed in the recess of the dock.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. An electronic docking system comprising:
a) a dock comprising a recess having at least one substantially vertically oriented side wall and a recess bottom face, wherein the dock comprises:
a pogo pin connector extending from the recess bottom face;
a first protrusion extending in a substantially perpendicular direction from the recess bottom face; and
a second protrusion extending in a substantially perpendicular direction from the recess bottom face; and
b) an electronic device comprising:
an electrical contact configured to contact the pogo pin connector;
a first alignment slot configured to accept the first protrusion; and
a second alignment slot configured to accept the second protrusion;
wherein at least a portion of the exterior perimeter of the recess comprises a rounded or beveled edge.

2. The electronic docking system of claim 1, further comprising at least one of:
one or more magnets within the recess; and
one or more magnets inside or coupled with the electronic device and configured to mate with the one or more magnets in the recess.

3. The electronic docking system of claim 1, wherein the dock further comprises at least one of an indicator, a motion sensor, and an ambient light sensor.

4. The electronic docking system of claim 1, wherein the electronic device further comprises at least one of an indicator, a motion sensor , and an ambient light sensor.

5. The electronic docking system of claim 1, wherein the pogo pin connector comprises 5 pogo pins.

6. The electronic docking system of claim 1, further comprising at least one support protrusion extending vertically from a perimeter edge of the recess of the dock.

7. The electronic docking system of claim 3 or 4, wherein the indicator indicates a charging mode corresponding to a transfer of energy from the pogo pin connector to the electronic device.

8. The electronic docking system of claim 7, wherein the indicator is further activated or deactivated by at least one of:
the motion sensor; and
the ambient light sensor.

9. The electronic docking system of claim 1, wherein the recess is generally rectangular and wherein the at least one vertically oriented side wall comprises four side walls.

10. The electronic docking system of claim 9, wherein the at least one substantially vertically oriented side wall in the recess of the dock is tapered away from at least one of the first protrusion and the second protrusion.

11. The electronic docking system of claim 9 or 10, wherein the recess of the dock is configured to guide an insertion of an edge of the electronic device such that the electrical contact of the electronic device accurately aligns with the pogo pin connector of the dock.

12. The electronic docking system of claim 9 or 10, wherein the recess of the dock is configured to guide an insertion of an edge of the electronic device such that the first and second alignment slots of the electronic device accurately aligns with the first and second protrusions extending from the recess bottom face.

13. The electronic docking system of claim 1, wherein at least one of the first protrusion and the second protrusion is oblong in shape.

14. The electronic docking system of claim 12, wherein the first and second protrusion extending from the recess bottom face have tapered or rounded side walls.

15. A docking station for an electronic system comprising:
a recess having at least one substantially vertically oriented side wall and a recess bottom face;
a pogo pin connector extending from the recess bottom face;
a first protrusion extending in a substantially perpendicular direction from the recess bottom face;
a second protrusion extending in a substantially perpendicular direction from the recess bottom face; and
at least one support protrusion configured to provide support to an electronic device when placed in the recess of the docking station.

16. The docking station of claim 15, further comprising at least one magnet within the recess configured to mate with one or more corresponding magnets inside or coupled with the electronic device when the electronic device is placed in the recess.

17. The docking station of claim 15, further comprising at least one of an indicator, a motion sensor, and an ambient light sensor.

18. The electronic docking system of claim 17, wherein the motion sensor is a combination sensor also configurable as an ambient light sensor.

19. The docking station of claim 16, wherein the indicator is further activated or deactivated by at least one of:
the motion sensor; and
the ambient light sensor.

20. The docking station of claim 15, wherein the pogo pin connector comprises 5 pogo pins.

21. The docking station of claim 15, wherein the at least one support protrusion is configured to provide support to the electronic device when placed in the recess of the docking station.

22. The docking station of claim 17, wherein the indicator indicates a charging mode corresponding to a transfer of energy from the pogo pin connector to the electronic device.

23. The docking station of claim 15, wherein the recess is generally rectangular and wherein the at least one vertically oriented side wall comprises four side walls.

24. The docking station of claim 23, wherein the at least one vertically oriented side wall in the recess of the dock is tapered away from the first protrusion and the second protrusion.

25. The docking station of claim 23 or 24, wherein the recess of the dock is configured to guide an insertion of an edge of the electronic device such that an electrical contact of the electronic device accurately aligns with the pogo pin connector of the dock.

26. The docking station of claim 23 or 24, wherein the recess of the dock is configured to guide an insertion of an edge of the electronic device such that a first alignment slot and a second alignment slot of the electronic device accurately aligns with the first protrusion and the second protrusion extending from the bottom recess face.

27. The docking station of claim 15, wherein at least one of the first protrusion and the second protrusion is oblong in shape.

28. The electronic docking system of claim 1, wherein the electronic device comprises at least one of:
laptop computers;
notebook computers;
sub-notebook computers;
netbook computers;
netpad computers;
media-streaming devices;
handheld computers;
Internet appliances;
mobile smartphones;
tablet computers;
disposable tablet computers;
personal digital assistants; and
video game consoles.

29. The electronic docking system of claim 28, wherein tablet computers comprise at least one of:
a booklet;
a slate; and
a convertible;
configuration.

30. The docking station of claim 15, wherein the electronic device comprises at least one of:
laptop computers;
notebook computers;
sub-notebook computers;
netbook computers;
netpad computers;
media-streaming devices;
handheld computers;
Internet appliances;
mobile smartphones;
tablet computers;
disposable tablet computers;
personal digital assistants; and
video game consoles.

31. The docking station of claim 30, wherein tablet computers comprise at least one of:
a booklet;
a slate; and
a convertible;
configuration.

32. The electronic docking system of claim 1, wherein the dock is configured to align the pogo pin connector and the electrical contact when the device is inserted into the dock at an angle, wherein an insertion angle of the electronic device into the dock is an angle between about −60 to +60 degrees from the substantially vertically oriented side wall extending from the recess bottom face.
